(19) United States
(12) Patent Application Publication
(10) Pub. No.: US 2005/0069453 A1
(43) Pub. Date: Mar. 31, 2005

(54) Methods for sterilizing preparations of urokinase

(76) Inventors: Ren-Yo Forng, Potomac, MD (US); David M. Mann, Gaithersburg, MD (US); Wilson Burgess, Clifton, VA (US); William N. Drohan, Springfield, VA (US); Martin J. MacPhee, North Potomac, MD (US); Shirley I. Miekka, Gaithersburg, MD (US)

Correspondence Address: FLESHNER & KIM, LLP, P.O. BOX 221200, CHANTILLY, VA, 20153, US

(21) Appl. No.: 10/672,068

(22) Filed: Sep. 29, 2003

(51) Int. Cl. A61L 2/00; C12N 9/72

(52) U.S. Cl. 422/23; 435/215

(57) ABSTRACT

Methods are disclosed for sterilizing preparations of urokinase to reduce the level therein of one or more active biological contaminants or pathogens, such as viruses, bacteria (including inter- and intracellular bacteria, such as mycoplasmas, ureaplasmas, nanobacteria, chlamydia and rickettsias), yeasts, molds, fungi, single or multicellular parasites, and prions or similar agents responsible, alone or in combination, for TSEs. These methods involve sterilizing preparations of urokinase with irradiation.

### BACKGROUND OF THE INVENTION

1. Field of the Invention

The present invention relates to methods for sterilizing preparations of urokinase to reduce the level therein of one or more active biological contaminants or pathogens, such as viruses, bacteria (including inter- and intracellular bacteria, such as mycoplasmas, ureaplasmas, nanobacteria, chlamydia, rickettsias), yeasts, molds, fungi, single or multicellular parasites and/or prions or similar agents responsible, alone or in combination, for TSEs. The present invention particularly relates to methods of sterilizing preparations of urokinase with irradiation.

2. Background of the Related Art

An important property of human blood is its ability to block lesions to the circulatory system by forming clots. Blood clotting is caused by a number of enzymes in the blood. These blood clotting enzymes lead to a proteolytic conversion of fibrinogen to fibrin using the enzyme thrombin. Fibrin then polymerizes with thrombocytes, erythrocytes and other blood components at the site of the lesion, thus forming a clot.

In addition, blood also contains a series of enzymes which counteract the clotting process and ensure blood flow; a process known as thrombolysis. The most important enzyme for thrombolysis is plasmin, which attacks the fibrin network and so causes dissolution of the clot. Plasmin is produced by proteolytic cleavage of the inactive precursor protein plasminogen by plasminogen activators. Endogenous human plasminogen activators include urokinase (u-plasminogen activator), and tissue plasminogen activator (t-plasminogen activator).

Cardiac infarcts and cerebral strokes are closely linked to the pathological formation of clots. In both types of infarct, clots are formed on the vessel walls under certain conditions—mostly as a result of arteriosclerotic alterations of the arteries. These clots can disturb the blood flow in the arteries, so that tissues can no longer be supplied with sufficient oxygen. After a cardiac infarct, a partial or complete necrosis of the heart muscle can occur. Correspondingly, blockage of cerebral arteries can also lead to severe damage of brain tissue.

Over the past twenty years, active treatment of myocardial infarct with thrombolytic agents has been proven to be both effective and efficient. In a number of studies, it was shown that treatments of patients suffering from myocardial infarct with streptokinase, anisoylated plasminogen-streptokinase activator complex (APSAC), two-chain urokinase (UK), recombinant single-chain urokinase (ptourokinase) or tissue plasminogen activator (tPA) lead to a significant reduction of mortality in comparison to non-treated patients. In order to improve the effectiveness of this type of therapy, a number of derivatives of tissue plasminogen activator and prourokinase have been synthesized using gene technology. Next to the aims of increasing the fibrinolytic activity and of reducing side-effects, of central interest is the search for forms suitable for bolus applications.

Plasminogen activators are employed as thrombolytic agents in the therapy of infarct patients, in order to start the dissolution of the clots by plasmin. At present, streptokinase, APSAC, UK, prourokinase and tPA are available for such therapies.

Urokinase, one type of plasminogen activator, is commonly used to dissolve blood clots that form in the heart, blood vessels or lungs after a heart attack or other disease process. Urokinase works best when it is given soon after the onset of heart attack symptoms and is used by injection into a vein usually when a blood clot seriously lessens the flow of blood to certain parts of the body. Urokinase is also used to dissolve blood clots that form in tubes that are placed in the body to allow treatment to be given over a long period of time, such as dialysis or injections into a vein.

Urokinase is a human protein which can be recovered from urine in two proteolytically active forms: high molecular weight urokinase (HUK) and low molecular weight urokinase (LUK). Urokinase is synthesized by various tissues as single-chain urokinase (prourokinase) which can be detected at low levels in human. The activated form of prourokinase has, as HUK, a molecular mass of 54 Kilodaltons, and contains three domains: an amino-terminal growth factor domain, a Kringle, and a serine protease domain. Although both prourokinase and plasminogen are present as proenzymes, prourokinase possesses an intrinsic activity which enables it to convert plasminogen into active plasmin.

Urokinase, however, has been subjected to shipment suspensions when inspections have indicated that the urokinase could have been infected with hepatitis B or mycoplasma. As such, substitute treatments had to be employed even though doctors expressed concern that the alternatives were less effective and had more potential to cause adverse effects than treatment with urokinase.

Since preparations of urokinase that are prepared for human, veterinary, diagnostic and/or experimental use may contain unwanted and potentially dangerous biologically active contaminants or pathogens, such as viruses, bacteria (including inter- and intracellular bacteria, such as mycoplasmas, ureaplasmas, nanobacteria, chlamydia and rickettsias), yeasts, molds, fungi, single or multicellular parasites, prions or similar agents responsible, alone or in combination, for TSEs, it is of utmost importance that any biologically active contaminant or pathogen in a preparation of urokinase be inactivated prior to use of the product. This is especially critical when the urokinase preparation is to be administered directly to a patient, for example, injected into a human vein, as discussed above. This is also critical for urokinase preparations that are prepared in media, since the media may contain prions, bacteria, viruses and/or other biological contaminants or pathogens.

Most procedures for producing preparations of urokinase have involved methods that screen or test the preparation for one or more particular biological contaminants or pathogens rather than removal or inactivation of the contaminant(s) and/or pathogen(s) from the preparation. Preparations that test positive for a biological contaminant or pathogen are merely not used. Examples of screening procedures include the testing for a particular virus in human blood from blood donors. Such procedures, however, are not always reliable and are not able to detect the presence of certain viruses, particularly in very low numbers. Additionally, such techniques are to no avail in the case of as yet unknown viruses or other contaminants or pathogens that may be present in blood. This reduces the value or certainty of the test in view of the consequences associated with a false negative result. False negative results can be life threatening in certain cases, for example in the case of Human Immunodeficiency Virus (HIV). Furthermore, in some instances it can take weeks, if not months, to determine whether or not the preparation is contaminated. Moreover, to date, there is no reliable test or assay for identifying prions within a preparation of urokinase that is suitable for screening out potential donors or infected material. This serves to heighten the need for an effective means of destroying prions within a preparation of urokinase, while still retaining the desired activity of that material. Therefore, it would be desirable to apply techniques that would kill or inactivate biological contaminants and pathogens during and/or after manufacturing the preparation of urokinase.

The importance of these techniques is apparent regardless of the source of the preparation of urokinase. All living cells and multi-cellular organisms can be infected with viruses and other pathogens. Thus the products of unicellular natural or recombinant organisms or tissues carry a risk of pathogen contamination. In addition to the risk that the producing cells or cell cultures may be infected, the processing of these and other preparations of urokinase creates opportunities for environmental contamination. The risks of infection are more apparent for multicellular natural and recombinant organisms, such as transgenic animals. Interestingly, even products from species as different from humans as transgenic plants carry risks, both due to processing contamination as described above, and from environmental contamination in the growing facilities, which may be contaminated by pathogens from the environment or infected organisms that co-inhabit the facility along with the desired plants. For example, a crop of transgenic corn grown out of doors, could be expected to be exposed to rodents such as mice during the growing season. Mice can harbour serious human pathogens such as the frequently fatal Hanta virus. Since these animals would be undetectable in the growing crop, viruses shed by the animals could be carried into the transgenic material at harvest. Indeed, such rodents are notoriously difficult to control, and may gain access to a crop during sowing, growth, harvest or storage. Likewise, contamination from overhead or perching birds has to potential to transmit such serious pathogens as the causative agent for psittacosis. Thus any preparation of urokinase, regardless of its source, may harbour serious pathogens that must be removed or inactivated prior to administration of the preparation to a recipient.

In conducting experiments to determine the ability of technologies to inactivate viruses, the actual viruses of concern ate seldom utilized. This is a result of safety concerns for the workers conducting the tests, and the difficulty and expense associated with the containment facilities and waste disposal. In their place, model viruses of the same family and class are used. In general, it is acknowledged that the most difficult viruses to inactivate are those with an outer shell made up of proteins, and that among these, the most difficult to inactivate are those of the smallest size. This has been shown to be true for gamma irradiation and most other forms of radiation as these viruses' diminutive size is associated with a small genome. The magnitude of direct effects of radiation upon a molecule are directly proportional to the size of the molecule. That is, the larger the target molecule, the greater the effect. As a corollary, it has been shown for gamma-irradiation that the smaller the viral genome, the higher the radiation dose required to inactive it.

Among the viruses that may contaminate both human and animal-derived preparations, the smallest, and thus most difficult to inactivate, belong to the family of Parvoviruses and the slightly larger protein-coated Hepatitis viruses. In humans, the Parvovirus B19, and Hepatitis A are agents of concern. In porcine-derived materials, the smallest corresponding virus is Porcine Parvovirus. Since this virus is harmless to humans, it is frequently chosen as a model virus for the human B19 Parvovirus. The demonstration of inactivation of this model parvovirus is considered adequate proof that the method employed will kill human B19 virus and Hepatitis A, and by extension, that it will also kill the larger and less hardy viruses such as HIV, CMV, Hepatitis B and C and others.

Previous efforts to render preparations safe for use have focussed on methods to remove or inactivate contaminants or pathogens in the products. Such methods include heat treating, filtration and the addition of chemical inactivants or sensitizers to the product.

Heat treatment requires that the product be heated to approximately 60° C. for periods as long as 70 hours which can be damaging to sensitive products. In some instances, heat inactivation can destroy 50% or more of the biological activity of the product.

Filtration involves filtering the product in order to physically remove contaminants. Unfortunately, this method may also remove products that have a high molecular weight. Further, the filter size may not be sufficiently small to remove small viruses and other similarly sized contaminants or pathogens, such as prions.

The procedure of chemical sensitization involves the addition of noxious agents which bind to the DNA/RNA of the virus and which are activated by radiation, such as UV. This radiation produces reactive intermediates and/or free radicals which bind to the DNA/RNA of the virus, break the chemical bonds in the backbone of the DNA/RNA, and/or cross-link or complex it in such a way that the virus can no longer replicate. According to such procedures, unbound sensitizer must be removed from the preparation prior to use, since the sensitizers are toxic and cannot be administered to a patient.

Irradiating a product with gamma radiation is another method of sterilizing a product. Gamma radiation is effective in destroying viruses and bacteria when given in high total doses (Keathly et al., “Is There Life After Irradiation? Part 2,” BioPharm July-August, 1993, and Leitman, Use of Blood Cell Irradiation in the Prevention of Post Transfusion Graft-vs-Host Disease,” Transfusion Science 10:219-239 (1989)). The published literature in this area, however, teaches that gamma radiation can be damaging to radiation sensitive products, such as blood, blood products, enzymes, protein and protein-containing products. In particular, it has been shown that high radiation doses are injurious to red cells, platelets and granulocytes (Leitman). U.S. Pat. No. 4,620,908 discloses that protein products must be frozen prior to irradiation in order to maintain the viability of the protein product. This patent concludes that “[i]f the gamma irradiation were applied while the protein material was at, for example, ambient temperature, the material would be also completely destroyed, that is the activity of the material would be rendered so low as to be virtually ineffective”. Unfortunately, many sensitive preparations containing urokinase may lose viability and activity if subjected to freezing for irradiation purposes and then thawing prior to administration to a patient.

In view of the difficulties discussed above, there remains a need for methods of sterilizing preparations of urokinase that are effective for reducing the level of active biological contaminants or pathogens without an adverse effect on the preparation.

### SUMMARY OF THE INVENTION

An object of the invention is to solve at least the above problems and/or disadvantages and to provide at least the advantages described hereinafter.

Accordingly, it is an object of the present invention to provide methods of sterilizing preparations of urokinase by reducing the level of active biological contaminants or pathogens without adversely affecting the preparation of urokinase. Other objects, features and advantages of the present invention will be set forth in the detailed description of preferred embodiments that follows, and in part will be apparent from the description or may be learned by practice of the invention. These objects and advantages of the invention will be realized and attained by the compositions and methods particularly pointed out in the written description and claims hereof.

According to these and other objects, a first embodiment of the present invention is directed to a method for sterilizing a preparation of urokinase that is sensitive to radiation, the method comprising irradiating the preparation of urokinase with radiation for a time effective to sterilize the preparation of urokinase at a rate effective to sterilize the preparation of urokinase and to protect the preparation of urokinase from the radiation.

Another embodiment of the present invention is directed to a method for sterilizing a preparation of urokinase that is sensitive to radiation, the method comprising: (i) applying to the preparation of urokinase at least one stabilizing process selected from the group consisting of: (a) adding to the preparation of urokinase at least one stabilizer; (b) reducing the residual solvent content of the preparation of urokinase; (c) reducing the temperature of the preparation of urokinase; (d) reducing the oxygen content of the preparation of urokinase; (e) adjusting or maintaining the pH of the preparation of urokinase; and (f) adding to the preparation of urokinase at least one non-aqueous solvent; and (ii) irradiating the preparation of urokinase with a suitable radiation at an effective rate for a time effective to sterilize the preparation of urokinase, wherein the at least one stabilizing process protects the preparation of urokinase from the radiation.

Another embodiment of the present invention is directed to methods for prophylaxis or treatment of a condition or disease in a mammal comprising introducing into a mammal in need thereof a preparation of urokinase sterilized according to the methods of the present invention.

Another embodiment of the present invention is directed to a preparation of urokinase and at least one stabilizer in an amount effective to preserve the preparation of urokinase for its intended use following sterilization with radiation.

Another embodiment of the present invention is directed to a preparation of urokinase sterilized according to the methods of the present invention.

Another embodiment of the present invention is directed to a preparation of urokinase, at least one non-aqueous solvent and/or at least one stabilizer in an amount effective to preserve the preparation of urokinase for its intended use following sterilization with radiation.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

The invention will be described in detail with reference to the following drawings in which like reference numerals refer to like elements wherein:

FIG. 1 illustrates the protective effects of the dipeptide stabilizer L-carnosine on irradiated LUK.

FIG. 2 illustrates the protective effects of the dipeptide stabilizer anserine on irradiated LUK.

FIG. 3 illustrates the protective effects of L-carnosine on irradiated LUK.

FIG. 4 illustrates the effects of gamma radiation on dried urokinase suspended in polypropylene glycol or phosphate buffered saline.

### DETAILED DESCRIPTION OF PREFERRED EMBODIMENTS

A. Definitions

Unless defined otherwise, all technical and scientific terms used herein are intended to have the same meaning as is commonly understood by one of ordinary skill in the relevant art.

As used herein, the singular forms “a,” “an,” and “the” include the plural reference unless the context clearly dictates otherwise.

As used herein, the term “preparation of urokinase” is intended to mean any preparation derived or obtained from a living organism that contains one or more plasminogen activators, either alone or in combination with one or more additional plasminogen activators or other compounds. Illustrative examples of plasminogen activators include, but are not limited to, the following: alteplase (recombinant), anistreplase, reteplase (recombinant), streptokinase, urokinase streptokinase, APSAC (anisoylated plasminogen-streptokinase activator complex), two-chain urokinase (UK), recombinant single-chain urokinase (recombinant prourokinase), high molecular weight urokinase, low molecular weight urokinase and tissue plasminogen activator (tPA), as well as variants, mutants and fragments of any one of these which retain the essential biological activity thereof.

As used herein, the term “sterilize” is intended to mean a reduction in the level of at least one active biological contaminant or pathogen found in the preparation being treated according to the present invention.

As used herein, the term “biological contaminant or pathogen” is intended to mean a biological contaminant or pathogen that, upon direct or indirect contact with a biological material, such as a preparation of urokinase, may have a deleterious effect on the biological material or upon a recipient thereof. Such biological contaminants or pathogens include the various viruses, bacteria, in both vegetative and spore states, (including inter- and intracellular bacteria, such as mycoplasmas, ureaplasmas, nanobacteria, chlamydia, rickettsias), yeasts, molds, fungi, prions or similar agents responsible, alone or in combination, for TSEs and/or single or multicellular parasites known to those of skill in the art to generally be found in or infect biological materials. Examples of other biological contaminants or pathogens include, but are not limited to, the following: viruses, such as human immunodeficiency viruses and other retroviruses, herpes viruses, filoviruses, circoviruses, paramyxoviruses, cytomegaloviruses, hepatitis viruses (including hepatitis A, B, C, and D variants thereof, among others), pox viruses, toga viruses, Ebstein-Barr viruses and parvoviruses; bacteria, such as Escherichia, Bacillus, Campylobacter, Streptococcus and Staphylococcus; nanobacteria; parasites, such as Trypanosoma and malarial parasites, including Plasmodium species; yeasts; molds; fungi; mycoplasmas and ureaplasmas; chlamydia; rickettsias, such as Coxiella burnetti; and prions and similar agents responsible, alone or in combination, for one or more of the disease states known as transmissible spongiform encephalopathies (TSEs) in mammals, such as scrapie, transmissible mink encephalopathy, chronic wasting disease (generally observed in mule deer and elk), feline spongiform encephalopathy, bovine spongiform encephalopathy (mad cow disease), Creutzfeld-Jakob disease (including variant CJD), Fatal Familial Insomnia, Gerstmann-Straeussler-Scheinker syndrome, kuru and Alpers syndrome. Further, as used herein, the term “active biological contaminant or pathogen” is intended to mean a biological contaminant or pathogen that is capable of causing a deleterious effect, either alone or in combination with another factor, such as a second biological contaminant or pathogen or a native protein (wild-type or mutant) or antibody, in a biological material, such as urokinase, and/or a recipient thereof.

As used herein, the term “a biologically compatible solution” is intended to mean a solution to which a biological material, such as urokinase, may be exposed, such as by being suspended or dissolved therein, and retain its essential biological and physiological characteristics. Such solutions may be of any suitable pH, tonicity, concentration and/or ionic strength.

As used herein, the term “a biologically compatible buffered solution” is intended to mean a biologically compatible solution having a pH and osmotic properties (e.g., tonicity, osmolality and/or oncotic pressure) suitable for maintaining the integrity of the material(s) therein, such as urokinase. Suitable biologically compatible buffered solutions typically have a pH between 2 and 8.5 and are isotonic or only moderately hypotonic or hypertonic. Biologically compatible buffered solutions are known and readily available to those of skill in the art. Greater or lesser pH and/or tonicity may also be used in certain applications. The ionic strength of the solution may be high or low, but is typically similar to the environments in which the preparation of urokinase is intended to be used.

As used herein, the term “stabilizer” is intended to mean a compound or material that, alone and/or in combination, reduces damage to the biological material being irradiated to a level that is insufficient to preclude the safe and effective use of the material. Illustrative examples of stabilizers that are suitable for use include, but are not limited to, the following, including structural analogs and derivatives thereof: antioxidants; free radical scavengers, including spin traps, such as tert-butyl-nitrosobutane (tNB), a-phenyl-tert-butylnitrone (PBN), 5,5-dimethylpyrroline-N-oxide (DMPO), tert-butylnitrosobenzene (BNB), a-(4-pyridyl-1-oxide)-N-tert-butylnitrone (4-POBN) and 3,5-dibromo-4-nitrosobenzenesulphonic acid (DBNBS); combination stabilizers, i.e., stabilizers which are effective at quenching both Type I and Type II photodynamic reactions; and ligands, ligand analogs, substrates, substrate analogs, modulators, modulator analogs, stereoisomers, inhibitors, and inhibitor analogs, such as hepatin, that stabilize the molecule(s) to which they bind. Preferred examples of additional stabilizers include, but are not limited to, the following: fatty acids, including 6,8-dimetcapto-octanoic acid (lipoic acid) and its derivatives and analogues (alpha, beta, dihydro, bisno and tetranot lipoic acid), thioctic acid, 6,8-dimercaptooctanoic acid, dihydrolopoate (DL-6,8-dithioloctanoic acid methyl ester), lipoamide, bisonot methyl ester and tetranot-dihydrolipoic acid, omega-3 fatty acids, omega-6 fatty acids, omega-9 fatty acids, furan fatty acids, oleic, linoleic, linolenic, arachidonic, eicosapentaenoic (EPA), docosahexaenoic (DHA), and palmitic acids and their salts and derivatives; carotenes, including alpha-, beta-, and gamma-carotenes; Co-Q10; xanthophylls; sucrose, polyhydric alcohols, such as glycerol, mannitol, inositol, and sorbitol; sugars, including derivatives and stereoisomers thereof, such as xylose, glucose, ribose, mannose, fructose, erythrose, threose, idose, arabinose, lyxose, galactose, allose, altrose, gulose, talose, and trehalose; amino acids and derivatives thereof, including both D- and L-forms and mixtures thereof, such as arginine, lysine, alanine, valine, leucine, isoleucine, proline, phenylalanine, glycine, serine, threonine, tyrosine, asparagine, glutamine, aspartic acid, histidine, N-acetylcysteine (NAC), glutamic acid, tryptophan, sodium captyl N-acetyl tryptophan, and methionine; azides, such as sodium azide; enzymes, such as Superoxide Dismutase (SOD), Catalase, and Δ4, Δ5 and Δ6 desaturases; uric acid and its derivatives, such as 1,3-dimethyluric acid and dimethylthiourea; allopurinol; thiols, such as glutathione and reduced glutathione and cysteine; trace elements, such as selenium, chromium, and boron; vitamins, including their precursors and derivatives, such as vitamin A, vitamin C (including its derivatives and salts such as sodium ascorbate and palmitoyl ascorbic acid) and vitamin E (and its derivatives and salts such as alpha-, beta-, gamma-, delta-, epsilon-, zeta-, and eta-tocopherols, tocopherol acetate and alpha-tocotrienol); chromanol-alpha-C6; 6-hydroxy-2,5,7,8-tetramethylchroma-2 carboxylic acid (Trolox) and derivatives; extraneous proteins, such as gelatin and albumin; tris-3-methyl-1-phenyl-2-pyrazolin-5-one (MCI-186); citiolone; puercetin; chrysin; dimethyl sulfoxide (DMSO); piperazine diethanesulfonic acid (PIPES); imidazole; methoxypsoralen (MOPS); 1,2-dithiane-4,5-diol; reducing substances, such as butylated hydroxyanisole (BHA) and butylated hydroxytoluene (BHT); cholesterol, including derivatives and its various oxidized and reduced forms thereof, such as low density lipoprotein (LDL), high density lipoprotein (HDL), and very low density lipoprotein (VLDL); probucol; indole derivatives; thimerosal; lazaroid and tirilazad mesylate; proanthenols; proanthocyanidins; ammonium sulfate; Pegorgotein (PEG-SOD); N-tert-butyl-alpha-phenylnitrone (PBN); 4-hydroxy-2,2,6,6-tetramethylpiperidin-1-oxyl (Tempol); mixtures of ascorbate, urate and Trolox C (Asc/urate/Trolox C); proteins, such as albumin, and peptides of two or more amino acids, any of which may be either naturally occurring amino acids, i.e., L-amino acids, or non-naturally occurring amino acids, i.e., D-amino acids, and mixtures, derivatives, and analogs thereof, including, but not limited to, arginine, lysine, alanine, valine, leucine, isoleucine, proline, phenylalanine, glycine, histidine, glutamic acid, tryptophan (Trp), serine, threonine, tyrosine, asparagine, glutamine, aspartic acid, cysteine, methionine, and derivatives thereof, such as N-acetylcysteine (NAC) and sodium capryl N-acetyl tryptophan, as well as homologous dipeptide stabilizers (composed of two identical amino acids), including such naturally occurring amino acids, as Gly-Gly (glycylglycine) and Trp-Ttp, and heterologous dipeptide stabilizers (composed of different amino acids), such as carnosine (β-alanyl-histidine), anserine (β-alanyl-methylhistidine), and Gly-Trp; and flavonoids/flavonols, such as diosmin, quercetin, rutin, silybin, silidianin, silicristin, silymarin, apigenin, apiin, chrysin, morin, isoflavone, flavoxate, gossypetin, myricetin, biacalein, kaempferol, curcumin, proanthocyanidin B2-3-O-gallate, epicatechin gallate, epigallocatechin gallate, epigallocatechin, gallic acid, epicatechin, dihydroquercetin, quercetin chalcone, 4,4′-dihydroxy-chalcone, isoliquiritigenin, phloretin, coumestrol, 4′,7-dihydroxy-flavanone, 4′,5-dihydroxy-flavone, 4′,6-dihydroxy-flavone, luteolin, galangin, equol, biochanin A, daidzein, formononetin, genistein, amentoflavone, bilobetin, taxifolin, delphinidin, malvidin, petunidin, pelargonidin, malonylapiin, pinosylvin, 3-methoxyapigenin, leucodelphinidin, dihydrokaempferol, apigenin 7-O-glucoside, pycnogenol, aminoflavone, purpurogallin fisetin, 2′,3′-dihydroxyflavone, 3-hydroxyflavone, 3′,4′-dihydroxyflavone, catechin, 7-flavonoxyacetic acid ethyl ester, catechin, hesperidin, and naringin. Particularly preferred examples include single stabilizers or combinations of stabilizers that are effective at quenching both Type I and Type II photodynamic reactions, and volatile stabilizers, which can be applied as a gas and/or easily removed by evaporation, low pressure, and similar methods. Additional preferred examples for use in the methods of the present invention include hydrophobic stabilizers. Other preferred stabilizers according to the methods of the present invention include compounds capable of preventing the generation of free radicals during irradiation. Other preferred stabilizers according to the methods of the present invention include compounds capable of preventing the generation of reactive oxygen species during irradiation. Still other preferred stabilizers according to the present invention include α-keto acids, including but not limited to lactate and pyruvate.

As used herein, the term “residual solvent content” is intended to mean the amount or proportion of freely-available liquid in the preparation of urokinase. Freely-available liquid means the liquid, such as water and/or an organic solvent (e.g., ethanol, isopropanol, polyethylene glycol, etc.), present in the preparation of urokinase being sterilized that is not bound to or complexed with one or more of the non-liquid components of the preparation of urokinase. Freely-available liquid includes intracellular water and/or other solvents. The residual solvent contents related as water referenced herein refer to levels determined by the FDA approved, modified Karl Fischer method (Meyer and Boyd, Analytical Chem., 31:215-219, 1959; May, et al., J. Biol. Standardization, 10:249-259, 1982; Centers for Biologics Evaluation and Research, FDA, Docket No. 89D-0140, 83-93; 1990) or by near infrared spectroscopy. Quantitation of the residual levels of water or other solvents may be determined by means well known in the art, depending upon which solvent is employed. The proportion of residual solvent to solute may also be considered to be a reflection of the concentration of the solute within the solvent. When so expressed, the greater the concentration of the solute, the lower the amount of residual solvent.

As used herein, the term “sensitizer” is intended to mean a substance that selectively targets viruses, bacteria, in both vegetative and spore states, (including inter- and intracellular bacteria, such as mycoplasmas, ureaplasmas, nanobacteria, chlamydia, rickettsias), yeasts, molds, fungi, single or multicellular parasites, and/or prions or similar agents responsible, alone or in combination, for TSEs, rendering them more sensitive to inactivation by radiation, therefore permitting the use of a lower rate or dose of radiation and/or a shorter time of irradiation than in the absence of the sensitizer. Illustrative examples of suitable sensitizers include, but are not limited to, the following: psoralen and its derivatives and analogs (including 3-carboethoxy psoralens); inactines and their derivatives and analogs; angelicins, khellins and coumarins which contain a halogen substituent and a water solubilization moiety, such as quaternary ammonium ion or phosphonium ion; nucleic acid binding compounds; brominated hematoporphyrin; phthalocyanines; purpurins; porphyrins; halogenated or metal atom-substituted derivatives of dihematoporphyrin esters, hematoporphyrin derivatives, benzoporphyrin derivatives, hydrodibenzoporphyrin dimaleimade, hydrodibenzoporphyrin, dicyano disulfone, tetracarbethoxy hydrodibenzoporphyrin, and tetracarbethoxy hydrodibenzoporphyrin dipropionamide; doxorubicin and daunomycin, which may be modified with halogens or metal atoms; netropsin; BD peptide, S2 peptide; S-303 (ALE compound); dyes, such as hypericin, methylene blue, eosin, fluoresceins (and their derivatives), flavins, merocyanine 540; photoactive compounds, such as bergapten; and SE peptide. In addition, atoms which bind to prions, and thereby increase their sensitivity to inactivation by radiation, may also be used. An illustrative example of such an atom would be the Copper ion, which binds to the prion protein and, with a Z number higher than the other atoms in the protein, increases the probability that the prion protein will absorb energy during irradiation, particularly gamma irradiation.

As used herein, the term “radiation” is intended to mean radiation of sufficient energy to sterilize at least some component of the irradiated preparation of urokinase. Types of radiation include, but are not limited to, the following: (i) corpuscular (streams of subatomic particles such as neutrons, electrons, and/or protons); and (ii) electromagnetic (originating in a varying electromagnetic field, such as radio waves, visible (both mono and polychromatic) and invisible light, infrared, ultraviolet radiation, x-ray radiation, gamma rays and mixtures thereof. Such radiation is often described as either ionizing (capable of producing ions in irradiated materials) radiation, such as gamma rays, and non-ionizing radiation, such as visible light. The sources of such radiation may vary and, in general, the selection of a specific source of radiation is not critical provided that sufficient radiation is given in an appropriate time and at an appropriate rate to effect sterilization. In practice, gamma radiation is usually produced by isotopes of Cobalt or Cesium, while X-rays are usually produced by machines that emit X-ray radiation, and electrons are often used to sterilize materials in a method known as “E-beam” irradiation that usually involves their production via a machine. Visible light, both mono- and polychromatic, is produced by machines and may, in practice, be combined with invisible light, such as infrared and UV, that is produced by the same machine or a different machine.

As used herein, the term “to protect” is intended to mean to reduce any damage to the preparation of urokinase being irradiated that would otherwise result from the irradiation of that material to a level that is insufficient to preclude the safe and effective use of the material following irradiation. In other words, a substance or process “protects” a preparation of urokinase from radiation if the presence of that substance or carrying out that process results in less damage to the material from irradiation than in the absence of that substance or process. Thus, a preparation of urokinase may be used safely and effectively after irradiation in the presence of a substance or following performance of a process that “protects” the material, but could not be used safely and effectively after irradiation under identical conditions but in the absence of that substance or the performance of that process.

As used herein, an “acceptable level” of damage may vary depending upon certain features of the particular method(s) of the present invention being employed, such as the nature and characteristics of the particular urokinase and/or non-aqueous solvent(s) being used, and/or the intended use of the material being irradiated, and can be determined empirically by one skilled in the art. An “unacceptable level” of damage would therefore be a level of damage that would preclude the safe and effective use of the biological material, such as urokinase, being sterilized. The particular level of damage in a given biological material may be determined using any of the methods and techniques known to one skilled in the art.

As used herein, the term “non-aqueous solvent” is intended to mean any liquid other than water in which a biological material, such as urokinase, may be dissolved or suspended or which may be disposed within a biological material, such as urokinase, and includes both inorganic solvents and, more preferably, organic solvents. Illustrative examples of suitable non-aqueous solvents include, but are not limited to, the following: alkanes and cycloalkanes, such as pentane, 2-methylbutane (isopentane), heptane, hexane, cyclopentane and cyclohexane; alcohols, such as methanol, ethanol, 2-methoxyethanol, isopropanol, n-butanol, t-butyl alcohol, and octanol; esters, such as ethyl acetate, 2-methoxyethyl acetate, butyl acetate and benzyl benzoate; aromatics, such as benzene, toluene, pyridine, xylene; ethers, such as diethyl ether, 2-ethoxyethyl ether, ethylene glycol dimethyl ether and methyl t-butyl ether; aldehydes, such as formaldehyde and glutaraldehyde; ketones, such as acetone and 3-pentanone (diethyl ketone); glycols, including both monomeric glycols, such as ethylene glycol and propylene glycol, and polymeric glycols, such as polyethylene glycol (PEG) and polypropylene glycol (PPG), e.g., PPG 400, PPG 1200 and PPG 2000; acids and acid anhydrides, such as formic acid, acetic acid, trifluoroacetic acid, phosphoric acid and acetic anhydride; oils, such as cottonseed oil, peanut oil, culture media, polyethylene glycol, poppyseed oil, safflower oil, sesame oil, soybean oil and vegetable oil; amines and amides, such as piperidine, N,N-dimethylacetamide and N,N-deimethylformamide; dimethylsulfoxide (DMSO); nitriles, such as benzonitrile and acetonitrile; hydrazine; detergents, such as polyoxyethylenesorbitan monolaurate (Tween 20) and monooleate (Tween 80), Triton and sodium dodecyl sulfate; carbon disulfide; halogenated solvents, such as dichloromethane, chloroform, carbon tetrachloride, 1,2-dichlorobenzene, 1,2-dichloroethane, tetrachloroethylene and 1-chlorobutane; furans, such as tetrahydrofuran; oxanes, such as 1,4-dioxane; and glycerin/glycerol. Particularly preferred examples of suitable non-aqueous solvents include non-aqueous solvents which also function as stabilizers, such as ethanol and acetone.

As used herein, the term “constant,” with respect to the rate of irradiation, is intended to include any variation in the rate of irradiation that results from natural decay of the source material over the duration of the sterilization procedure.

As used herein, the term “not constant,” with respect to the rate of irradiation, is intended to mean that the variation in the rate of irradiation is greater than any variation in the rate of irradiation that results from natural decay of the source material over the duration of the sterilization procedure.

B. Particularly Preferred Embodiments

A first preferred embodiment of the present invention is directed to a method for sterilizing a preparation of urokinase that is sensitive to radiation, the method comprising irradiating the preparation of urokinase with radiation for a time effective to sterilize the preparation of urokinase at a rate effective to sterilize the preparation of urokinase and to protect the preparation of urokinase from the radiation.

A second preferred embodiment of the present invention is directed to a method for sterilizing a preparation of urokinase that is sensitive to radiation, the method comprising: (i) applying to the preparation of urokinase at least one stabilizing process selected from the group consisting of (a) adding to the preparation of urokinase at least one stabilizer; (b) reducing the residual solvent content of the preparation of urokinase; (c) reducing the temperature of the preparation of urokinase; (d) reducing the oxygen content of the preparation of urokinase; (e) adjusting or maintaining the pH of the preparation of urokinase; and (f) adding to the preparation of urokinase at least one non-aqueous solvent; and (ii) irradiating the preparation of urokinase with a suitable radiation at an effective rate for a time effective to sterilize the preparation of urokinase, wherein the at least one stabilizing process protects the preparation of urokinase from the radiation.

A third preferred embodiment of the present invention is directed to methods for prophylaxis or treatment of a condition or disease in a mammal comprising introducing into a mammal in need thereof a preparation of urokinase sterilized according to the methods of the present invention.

A fourth preferred embodiment of the present invention is directed to a preparation of urokinase and at least one stabilizer in an amount effective to preserve the preparation of urokinase for its intended use following sterilization with radiation.

A fifth preferred embodiment of the present invention is directed to a preparation of urokinase sterilized according to the methods of the present invention.

Another preferred embodiment of the present invention is directed to a preparation of urokinase, at least one non-aqueous solvent and/or at least one stabilizer in an amount effective to preserve the preparation of urokinase for its intended use following sterilization with radiation.

The non-aqueous solvent is preferably a non-aqueous solvent that is not prone to the formation of free-radicals upon irradiation, and more preferably a non-aqueous solvent that is not prone to the formation of free-radicals upon irradiation and that has little or no dissolved oxygen or other gas(es) that is (are) prone to the formation of free-radicals upon irradiation. Volatile non-aqueous solvents are particularly preferred, even more particularly preferred are non-aqueous solvents that are stabilizers, such as ethanol and acetone.

According to certain embodiments of the present invention, the preparation of urokinase may contain a mixture of water and a non-aqueous solvent, such as ethanol and/or acetone. In such embodiments, the non-aqueous solvent(s) is (are) preferably a non-aqueous solvent that is not prone to the formation of free-radicals upon irradiation, and most preferably a non-aqueous solvent that is not prone to the formation of free-radicals upon irradiation and that has little or no dissolved oxygen or other gas(es) that is (are) prone to the formation of free-radicals upon irradiation. Volatile non-aqueous solvents are particularly preferred, even more particularly preferred are non-aqueous solvents that are also stabilizers, such as ethanol and acetone.

According to certain methods of the present invention, at least one stabilizer is preferably added prior to irradiation of the preparation of urokinase. The at least one stabilizer is preferably added to the preparation of urokinase in an amount that is effective to protect the preparation of urokinase from the radiation. Alternatively, the stabilizer is added to the preparation of urokinase in an amount that, together with a non-aqueous solvent or the effective rate, is effective to protect the preparation of urokinase from the radiation. Suitable amounts of stabilizer may vary depending upon certain features of the particular method(s) of the present invention being employed, such as the particular stabilizer being used and/or the nature and characteristics of the particular urokinase being irradiated and/or its intended use, and can be determined empirically by one skilled in the art.

According to certain preferred embodiments of the present invention, the preparation of urokinase contains at least one stabilizer in a concentration of at least 1 nM, at least 1 mM, at least 2 mM, at least 5 mM, at least 10 mM, at least 25 mM, at least 50 mM, at least 100 mM or at least 200 mM. Preferred ranges of stabilizer concentrations include, but are not limited to, from 0.1 mM to 10 mM, from 0.1 to 50 mM, from 10 to 100 mM, from 10 mM to 50 mM and from 50 to 100 mM. In certain preferred embodiments of the present invention, the preparation of urokinase contains at least one stabilizer in a concentration of about 250 mM.

According to certain methods of the present invention, the residual solvent content of the preparation of urokinase is preferably reduced prior to irradiation of the preparation of urokinase with radiation. The residual solvent content is reduced to a level that is effective to protect the preparation of urokinase from the radiation. Suitable levels of residual solvent content may vary depending upon certain features of the particular method(s) of the present invention being employed, such as the nature and characteristics of the particular preparation of urokinase and/or stabilizer being used, and/or the intended use of the preparation of urokinase being irradiated, and can be determined empirically by one skilled in the art. There may be preparations for which it is desirable to maintain the residual solvent content to within a particular range, rather than a specific value, for example when the solvent, or at least one of the solvents in a mixture, is also a stabilizer, such as an alcohol (e.g. ethanol) or dialkyl ketone (e.g. acetone).

When the preparation of urokinase is in a liquid or solid phase, and particularly preferably when the solvent is water, the residual solvent content is generally less than about 15%, typically less than about 10%, more typically less than about 9%, even more typically less than about 8%, usually less than about 5%, preferably less than about 3.0%, more preferably less than about 2.0%, even more preferably less than about 1.0%, still more preferably less than about 0.5%, still even more preferably less than about 0.2% and most preferably less than about 0.08%.

The solvent may preferably be a non-aqueous solvent, more preferably a non-aqueous solvent that is not prone to the formation of free-radicals upon irradiation, and most preferably a non-aqueous solvent that is not prone to the formation of free-radicals upon irradiation and that has little or no dissolved oxygen or other gas(es) that is (are) prone to the formation of free-radicals upon irradiation. Volatile non-aqueous solvents are particularly preferred, even more particularly preferred are non-aqueous solvents that are stabilizers, such as ethanol and acetone.

In certain embodiments of the present invention, the solvent may be a mixture of water and a non-aqueous solvent or solvents, such as ethanol and/or acetone. In such embodiments, the non-aqueous solvent(s) is preferably a non-aqueous solvent that is not prone to the formation of free-radicals upon irradiation, and most preferably a non-aqueous solvent that is not prone to the formation of free-radicals upon irradiation and that has little or no dissolved oxygen or other gas(es) that is (are) prone to the formation of free-radicals upon irradiation. Volatile non-aqueous solvents are particularly preferred, even more particularly preferred are non-aqueous solvents that are stabilizers, such as ethanol and acetone.

In a preferred embodiment, when the residual solvent is water, the residual solvent content of a preparation of urokinase is reduced by dissolving or suspending the preparation of urokinase in a non-aqueous solvent that is capable of dissolving in water. Preferably, such a non-aqueous solvent is not prone to the formation of free-radicals upon irradiation and has little or no dissolved oxygen or other gas(es) that is (are) prone to the formation of free-radicals upon irradiation.

When the preparation of urokinase is in a liquid phase, reducing the residual solvent content may be accomplished by any of a number of means, such as by increasing the solute concentration. In this manner, the concentration of the preparation of urokinase dissolved within the solvent may be increased to generally at least about 0.5%, typically at least about 1%, usually at least about 5%, preferably at least about 10%, more preferably at least about 15%, even more preferably at least about 20%, still even more preferably at least about 25%, and most preferably at least about 50%.

The residual solvent content of a preparation of urokinase may be reduced by any of the methods and techniques known to those skilled in the art for reducing solvent from a preparation of urokinase without producing an unacceptable level of damage to the preparation. Such methods include, but are not limited to, evaporation, concentration, centrifugal concentration, vitrification, addition of solute, lyophilization (with or without the prior addition of at least one stabilizer, such as ascorbate) and spray-drying.

A particularly preferred method for reducing the residual solvent content of a preparation of urokinase is lyophilization, even more preferred is lyophilization following the addition of ascorbate.

In certain embodiments of the present invention, the residual solvent content of a particular preparation of urokinase may be found to lie within a range, rather than at a specific point. Such a range for the preferred residual solvent content of a particular preparation of urokinase may be determined empirically by one skilled in the art.

While not wishing to be bound by any theory of operability, it is believed that the reduction in residual solvent content reduces the degrees of freedom of the preparation of urokinase, reduces the number of targets for free radical generation and may restrict the solubility or diffusion of these free radicals. Similar results might therefore be achieved by lowering the temperature of the preparation of urokinase below its eutectic point or below its freezing point, or by vitrification to likewise reduce the degrees of freedom of the preparation of urokinase. These results may permit the use of a higher rate and/or dose of radiation than might otherwise be acceptable. Thus, the methods described herein may be carried out at any temperature that does not result in an unacceptable level of damage to the preparation. Preferably, the methods described herein are performed at ambient temperature or below ambient temperature, such as below the eutectic point or freezing point of the preparation of urokinase being irradiated.

In accordance with the methods of the present invention, an “acceptable level” of damage may vary depending upon certain features of the particular method(s) of the present invention being employed, such as the nature and characteristics of the particular preparation of urokinase and/or stabilizer being used, and/or the intended use of the preparation of urokinase being irradiated, and can be determined empirically by one skilled in the art. An “unacceptable level” of damage would therefore be a level of damage that would preclude the safe and effective use of the preparation of urokinase being sterilized. The particular level of damage in a given preparation of urokinase may be determined using any of the methods and techniques known to one skilled in the art.

According to certain methods of the present invention, the preparation of urokinase to be sterilized may be immobilized upon a solid surface by any means known and available to one skilled in the art. For example, the preparation of urokinase to be sterilized may be present as a coating or surface on a biological or non-biological substrate.

The radiation employed in the methods of the present invention may be any radiation effective for the inactivation of one or more biological contaminants or pathogens of the preparation of urokinase being treated. The radiation may be corpuscular, including E-beam radiation. Preferably the radiation is electromagnetic radiation, including visible light, infrared, x-ray radiation, UV light and mixtures of various wavelengths of electromagnetic radiation. A particularly preferred form of radiation is gamma radiation.

According to the methods of the present invention, the preparation of urokinase is irradiated with radiation at a rate effective to sterilize the preparation of urokinase, while not producing an unacceptable level of damage to the preparation of urokinase. Suitable rates of irradiation may vary depending upon certain features of the methods of the present invention being employed, such as the nature and characteristics of the particular preparation of urokinase, which may contain a non-aqueous solvent, being irradiated, the particular form of radiation involved, and/or the particular biological contaminants or pathogens being inactivated. Suitable rates of irradiation can be determined empirically by one skilled in the art. Preferably, the rate of irradiation is constant for the duration of the sterilization procedure. When this is impractical or otherwise not desired, a variable or discontinuous irradiation may be utilized.

According to the methods of the present invention, the rate of irradiation may be optimized to produce the most advantageous combination of product recovery and time required to complete the operation. Both low (<3 kGy/hour) and high (>3 kGy/hour) rates may be utilized in the methods described herein to achieve such results. Such rates may be used alone or in combination. The rate of irradiation is preferably selected to optimize the recovery of the urokinase while still sterilizing the preparation of urokinase. Although reducing the rate of irradiation may serve to decrease damage to the preparation of urokinase, it will also result in longer irradiation times being required to achieve a particular desired total dose. A higher dose rate may therefore be preferred in certain circumstances, such as to minimize logistical issues and costs, and may be possible particularly when used in accordance with the methods described herein for protecting preparation of urokinase from irradiation.

According to a particularly preferred embodiment of the present invention, the rate of irradiation is not more than 3.0 kGy/hour, more preferably between about 0.1 kGy/hr and 3.0 kGy/hr, even more preferably between about 0.25 kGy/hr and 2.0 kGy/hour, still even more preferably between about 0.5 kGy/hr and 1.5 kGy/hr and most preferably between about 0.5 kGy/hr and 1.0 kGy/hr. In other preferred embodiments of the present invention, the rate of irradiation is not more than 2.5 kGy/hr, more preferably not more than 2.0 kGy/hr, even more preferably not more than 1.0 kGy/hr and most preferably not more than 0.3 kGy/hr. Such rates may be used alone or in combination.

According to another particularly preferred embodiment of the present invention, the rate of irradiation is more than 3.0 kGy/hr, more preferably at least 5 kGy/hr, even more preferably at least 18 kGy/hr, even more preferably at least 30 kGy/hr and most preferably at least 45 kGy/hr or greater. Such rates may be used alone or in combination.

According to certain preferred embodiments of the present invention, the rate of irradiation is not constant for the duration of the sterilization procedure. According to such embodiments, the rate of irradiation may be increased and/or decreased over the duration of the sterilization procedure. For instance, the rate of irradiation may include a relatively low rate of irradiation, such as 0.3 kGy/hr, and be increased to a rate of irradiation greater than 0.3 kGy/hr. Subsequently, the rate of irradiation may be decreased. According to the methods of the present invention, the rate of irradiation may be decreased and/or increased any number of times suitable to achieve a desired total dose of radiation. Additionally, high and/or low rates may be employed according to such embodiments.

According to other preferred embodiments of the present invention, irradiation is discontinuous over the duration of the sterilization procedure. For example, irradiation may be carried out for an initial period, interrupted for a subsequent period and then continued. According to such embodiments any desired number of breaks in irradiation may be employed, so long as a desired total dose of irradiation is achieved. During the periods of irradiation, the rate of irradiation may be constant and/or not constant. Additionally, high and/or low rates may be employed according to such embodiments.

According to other preferred embodiments of the present invention, combinations of constant and not constant rates of irradiation may be used over the duration of the sterilization procedure. For instance, over the duration of the sterilization procedure constant rates of irradiation may be used for one or more periods and not constant rates may be used for one or more other periods. Any number of constant and not constant rates may be used, as long as a desired total dose of radiation is achieved. Additionally, high and/or low rates may be employed according to such embodiments.

According to the methods of the present invention, the preparation of urokinase to be sterilized is irradiated with the radiation for a time effective for the sterilization of the preparation of urokinase. Combined with irradiation rate, the appropriate irradiation time results in the appropriate dose of irradiation being applied to the preparation of urokinase. Suitable irradiation times may vary depending upon the particular form and rate of radiation involved, the nature and characteristics of the particular preparation of urokinase being irradiated and/or the particular biological contaminants or pathogens being inactivated. Suitable irradiation times can be determined empirically by one skilled in the art.

According to the methods of the present invention, the urokinase to be sterilized is irradiated with radiation up to a total dose effective for the sterilization of the urokinase, while not producing an unacceptable level of damage to the urokinase. Suitable total doses of radiation may vary depending upon certain features of the methods of the present invention being employed, such as the nature and characteristics of the particular urokinase being irradiated, the particular form of radiation involved, and/or the particular biological contaminants or pathogens being inactivated. Suitable total doses of radiation can be determined empirically by one skilled in the art. Preferably, the total dose of radiation is at least 10 kGy, more preferably at least 25 kGy, even more preferably at least 30 kGy, and still more preferably at least 40 kGy or greater, such as 50 kGy, 55 kGy, 65 kGy, 70 kGy 80 kGy, 90 kGy, 105 kGy, 120 kGy or greater.

The particular geometry of the preparation of urokinase being irradiated, such as the thickness and distance from the source of radiation, may be determined empirically by one skilled in the art. A preferred embodiment is a geometry that provides for an even rate of irradiation throughout the preparation. A particularly preferred embodiment is a geometry that results in a short path length for the radiation through the preparation, thus minimizing the differences in radiation dose between the front and back of the preparation. This may be further minimized in some preferred geometries, particularly those wherein the preparation has a constant radius about its axis that is perpendicular to the radiation source, by the utilization of a means of rotating the preparation about said axis.

Similarly, according to certain methods of the present invention, an effective package for containing the preparation during irradiation is one which provides stability under the influence of irradiation, and which minimizes the interactions between the package and the radiation. Preferred packages maintain a seal against the external environment before, during and post-irradiation, and are not reactive with the preparation within, nor do they produce chemicals that may interact with the preparation within. Particularly preferred examples include but are not limited to containers that comprise glasses stable when irradiated, stoppered with stoppers made of rubber that is relatively stable during radiation and liberates a minimal amount of compounds from within, and sealed with metal crimp seals of aluminum or other suitable materials with relatively low Z numbers. Suitable materials can be determined by measuring their physical performance, and the amount and type of reactive leachable compounds post-irradiation and by examining other characteristics known to be important to the containment of preparation of urokinase empirically by one skilled in the art.

According to certain methods of the present invention, an effective amount of at least one sensitizing compound may optionally be added to the preparation of urokinase prior to irradiation, for example to enhance the effect of the irradiation on the biological contaminant(s) or pathogen(s) therein, while employing the methods described herein to minimize the deleterious effects of irradiation upon the preparation of urokinase. Suitable sensitizers are known to those skilled in the art, and include, for example, psoralens and their derivatives and analogs and inactines and their derivatives and analogs.

According to the methods of the present invention, the irradiation of the urokinase may occur at any temperature that is not deleterious to the urokinase being sterilized. According to one preferred embodiment, the urokinase being irradiated is at ambient temperature for at least a portion of the irradiation and preferably at the initiation of irradiation. According to an alternate preferred embodiment, the urokinase being irradiated is at reduced temperature, i.e., a temperature below ambient temperature, such as at least 4° C., at least 0° C., at least −10° C., at least −20° C., at least −30° C., at least −40° C., at least −50° C., at least −55° C., at least −60° C., at least −70° C., at least −72° C., at least dry ice temperature or at least −196° C., for at least a portion of the irradiation and preferably at the initiation of irradiation. According to this embodiment of the present invention, the urokinase being irradiated is preferably at or below the freezing or eutectic point(s) of the urokinase and/or the residual solvent therein, for at least a portion of the irradiation and preferably at the initiation of irradiation. Particularly preferred temperature ranges according to certain preferred embodiments of the present invention include, but are not limited to, from −30° C. to −50° C., from −30° C. to −70° C., from −10° C. to −30° C., from −10° C. to −50° C. and from −10° C. to −55° C., for at least a portion of the irradiation and preferably at the initiation of irradiation.

According to another alternate preferred embodiment, the urokinase being irradiated is at elevated temperature, i.e., a temperature above ambient temperature, such as 37° C., 60° C., 72° C. or 80° C., for at least a portion of the irradiation and preferably at the initiation of irradiation. While not wishing to be bound by any theory, the use of elevated temperature may enhance the effect of irradiation on the biological contaminant(s) or pathogen(s) and therefore allow the use of a lower total dose of radiation.

Most preferably, the irradiation of the preparation of urokinase occurs at a temperature that protects the preparation from radiation. Suitable temperatures can be determined empirically by one skilled in the art.

In certain embodiments of the present invention, the temperature at which irradiation is performed may be found to lie within a range, rather than at a specific point. Such a range for the preferred temperature for the irradiation of a particular preparation of urokinase may be determined empirically by one skilled in the art.

According to the methods of the present invention, the irradiation of the preparation of urokinase may occur at any pressure which is not deleterious to the preparation of urokinase being sterilized. According to one preferred embodiment, the preparation of urokinase is irradiated at elevated pressure. More preferably, the preparation of urokinase is irradiated at elevated pressure due to the application of sound waves, the use of a volatile, compression or other means known to those skilled in the art. While not wishing to be bound by any theory, the use of elevated pressure may enhance the effect of irradiation on the biological contaminant(s) or pathogen(s) and/or enhance the protection afforded by one or more stabilizers, and therefore allow the use of a lower total dose of radiation. Suitable pressures can be determined empirically by one skilled in the art.

Generally, according to the methods of the present invention, the pH of the preparation of urokinase undergoing sterilization is about 7. In some embodiments of the present invention, however, the preparation of urokinase may have a pH of less than 7, preferably less than or equal to 6, more preferably less than or equal to 5, even more preferably less than or equal to 4, and most preferably less than or equal to 3. In alternative embodiments of the present invention, the preparation of urokinase may have a pH of greater than 7, preferably greater than or equal to 8, more preferably greater than or equal to 9, even more preferably greater than or equal to 10, and most preferably greater than or equal to 11. According to certain embodiments of the present invention, the pH of the preparation undergoing sterilization is at or near the isoelectric point of the enzyme(s) contained in the preparation. According to other embodiments of the present invention, the pH of the preparation undergoing sterilization is at or near the pH at which at least one enzyme in the preparation has maximal affinity for its substrate(s). Suitable pH levels can be determined empirically by one skilled in the art.

Similarly, according to the methods of the present invention, the irradiation of the preparation of urokinase may occur under any atmosphere that is not deleterious to the preparation of urokinase being treated. According to one preferred embodiment, the preparation of urokinase is held in a low oxygen atmosphere or an inert atmosphere. When an inert atmosphere is employed, the atmosphere is preferably composed of a noble gas, such as helium or argon, more preferably a higher molecular weight noble gas, and most preferably argon. According to another preferred embodiment, the preparation of urokinase is held under vacuum while being irradiated. According to a particularly preferred embodiment of the present invention, a preparation of urokinase (lyophilized, liquid or frozen) is stored under vacuum or an inert atmosphere (preferably a noble gas, such as helium or argon, more preferably a higher molecular weight noble gas, and most preferably argon) prior to irradiation. According to an alternative preferred embodiment of the present invention, a liquid preparation of urokinase is held under low pressure, to decrease the amount of gas, particularly oxygen, dissolved in the liquid, prior to irradiation, either with or without a prior step of solvent reduction, such as lyophilization. Such degassing may be performed using any of the methods known to one skilled in the art.

In another preferred embodiment, where the preparation of urokinase contains oxygen or other gases dissolved within or associated with it, the amount of these gases within or associated with the preparation may be reduced by any of the methods and techniques known and available to those skilled in the art, such as the controlled reduction of pressure within a container (rigid or flexible) holding the preparation to be treated or by placing the preparation in a container of approximately equal volume.

It will be appreciated that the combination of one or more of the features described herein may be employed to further minimize undesirable effects upon the preparation of urokinase caused by irradiation, while maintaining adequate effectiveness of the irradiation process on the biological contaminant(s) or pathogen(s). For example, in addition to the use of a stabilizer, a particular preparation of urokinase may also be lyophilized, held at reduced temperature and kept under vacuum prior to irradiation to further minimize undesirable effects.

The sensitivity of a particular biological contaminant or pathogen to radiation is commonly calculated by determining the dose necessary to inactivate or kill all but 37% of the agent in a sample, which is known as the D₃₇ value. The desirable components of a preparation of urokinase may also be considered to have a D₃₇ value equal to the dose of radiation required to eliminate all but 37% of their desirable biological and physiological characteristics.

In accordance with certain preferred methods of the present invention, the sterilization of a preparation of urokinase is conducted under conditions that result in a decrease in the D₃₇ value of the biological contaminant or pathogen without a concomitant decrease in the D₃₇ value of the preparation of urokinase. In accordance with other preferred methods of the present invention, the sterilization of a preparation of urokinase is conducted under conditions that result in an increase in the D₃₇ value of the preparation of urokinase. In accordance with the most preferred methods of the present invention, the sterilization of a preparation of urokinase is conducted under conditions that result in a decrease in the D₃₇ value of the biological contaminant or pathogen and a concomitant increase in the D₃₇ value of the preparation of urokinase.

In accordance with certain preferred methods of the present invention, the sterilization of urokinase is conducted under conditions that reduce the possibility of the production of neo-antigens. In accordance with other preferred embodiments of the present invention, the sterilization of urokinase is conducted under conditions that result in the production of substantially no neo-antigens. The present invention also includes tissues sterilized according to such methods.

In accordance with certain preferred methods of the present invention, the sterilization of urokinase is conducted under conditions that reduce the total antigenicity of the tissue(s). In accordance with other preferred embodiments of the present invention the sterilization of urokinase is conducted under conditions that reduce the number of reactive allo-antigens and/or xeno-antigens in the tissue(s). The present invention also includes tissues sterilized according to such methods.

According to certain preferred embodiments of the present invention, urokinase sterilized according to the methods described herein may be introduced into a mammal in need thereof for prophylaxis or treatment of a condition or disease. Methods of introducing urokinase into a mammal are known to those skilled in the art.

According to certain preferred embodiments of the present invention, the functional activity of the preparation of urokinase following sterilization is about 100% of the pre-irradiation value. In other preferred embodiments of the present invention, the functional activity of the preparation of urokinase following sterilization is at least 95% of the pre-irradiation value, at least 90% of the pre-irradiation value, at least 85% of the pre-irradiation value, at least 80% of the pre-irradiation value, at least 70% of the pre-irradiation value, at least 60% of the pre-irradiation value or at least 50% of the pre-irradiation value.

Other preferred embodiments of the present invention are directed to compositions containing urokinase sterilized according to the methods disclosed herein. Such compositions include, but are not limited to, urokinase treated with a single or plurality of stabilizing processes. Additionally, such compositions may be further treated or processed and may contain additional additives, supplements and the like.

According to certain preferred embodiments of the present invention, the irradiation of the preparation is performed under conditions whereby the temperature of the preparation increases during the irradiation from an initial temperature (Tᵢ) to a final temperature (T_{f}). Preferably, the increase in the temperature of the preparation (ΔT) is about equal to the total dose of radiation (D) divided by the specific heat capacity (c) of the preparation. Specific heat capacities of particular preparations are known, or may be determined empirically by one skilled in the art using methods and techniques known in the art.

Preferably, the final temperature (T_{f}) is at or below a level effective to protect the preparation from the radiation. According to such embodiments, the maximum acceptable temperature (Tₘₐₓ) for a particular preparation is preferably determined empirically by one skilled in the art employing the particular irradiation conditions desired. According to such embodiments of the present invention, the initial temperature (Tᵢ) of the preparation is then preferably set at a level at or below Tₘₐₓ−ΔT prior to irradiation.

According to other embodiments of the present invention, the increase in the temperature of the preparation is less than the total dose of radiation (D) divided by the specific heat capacity (c) of the preparation. Such variation may be due to the particular preparation being irradiated, the size of the sample being irradiated, the packaging in which the sample is contained, the particular method(s) of cooling, as well as the environment in which the package is held during irradiation. According to such embodiments, the increase in the temperature of the preparation (ΔT) is preferably determined empirically by one skilled in the art using known methods and techniques. The initial temperature (Tᵢ) of the preparation is then preferably set at a level at or below Tₘₐₓ−ΔT prior to irradiation.

### EXAMPLES

The following examples are illustrative, but not limiting, of the present invention. Other suitable modifications and adaptations are of the variety normally encountered by those skilled in the art, and are fully within the spirit and scope of the present invention. It will be understood to those of ordinary skill in the art that the methods of the present invention can be carried out with a wide and equivalent range of conditions, formulations and other parameters without departing from the scope of the invention or any embodiments thereof. Unless otherwise noted, all gamma irradiation was accomplished using a ⁶⁰Co source.

### Example 1

In this experiment, the effect of gamma irradiation at various doses on liquid and dry low molecular weight urokinase was evaluated.

Method

Dry or liquid (1000 IU/ml) Sigma urokinase was irradiated to a total dose of 45 kGy at a dose rate of 30 kGy/hr or 0.6 kGy/hr with gamma irradiation and then assayed for structural integrity. Following irradiation, the samples were assayed using 500 IU/ml low molecular weight urokinase (LUK) and 1500 μM CalBiochem colorimetric substrate at room temperature. Readings were taken at 5 minutes, 25 minutes and 2 hours post-incubation.

Results

OD₂₈₀ of 1:10 dilution of dry samples showed less than 5% variation in protein concentration among all samples. For dry samples irradiated at 30 kGy/hr and 0.6 kGy/hr, the recovery was 91.3% and 65.3%, respectively. For liquid samples irradiated at 30 kGy/hr and 0.6 kGy/hr, the recovery was 57.9% and 48.3%, respectively.

### Example 2

In this experiment, the effect of Tris buffer and phosphate buffer on lyophilized LUK irradiated to a total dose of 45 kGy at a dose rate of 1.9 kGy/hr with gamma radiation was evaluated.

Method

Samples of 400 μl Sigma LUK (1,000 IU/ml, in H₂O) were prepared in the presence or absence of 200 mM sodium ascorbate in 3 ml glass vials. The samples included either 35 mM phosphate buffer (pH 7.5) or Tris buffer (pH 7.6). Following lyophilization, the samples were either not irradiated or irradiated to a total dose of 45 kGy at a dose rate of 1.9 kGy/hr with gamma radiation. The samples were then reconstituted with 4001 μl ddH₂O, and assayed in duplicate wells in a 96 well microtiter plate with 1500 μM CalBiochem urokinase colorimetric substrate #1 at room temperature. OD₄₀₅ and OD₆₂₀ were taken at 5 and 25 minute intervals after reaction.

Results

Recovery of samples irradiated to 45 kGy in the presence of sodium ascorbate and either Tris or phosphate buffer was about 90%. For samples lyophilized in the absence of sodium ascorbate, about 50% of the activity was lost with little additional loss of activity following irradiation to 45 kGy. Samples lyophilized in the absence of a buffer and ascorbate showed recovery of only about 2.5%.

### Example 3

In this experiment, the effect of gamma irradiation on liquid urokinase in the presence of varying concentrations of sodium ascorbate was evaluated.

Method

Samples of Sigma urokinase (50 μL at 1000 IU/ml) in the presence of varying concentrations of ascorbate (0 to 1000 mM) were irradiated to a total dose of either 0 or 45 kGy with gamma radiation. Irradiation was carried out at 4° C. at a dose rate of about 1.8 kGy/hr. Urokinase colorimetric substrate I was then added to each well to a final concentration of 500 μM (i.e., 50 μL of 1000 μM stock in 2× Assay buffer). Absorbance at 405 to 620 nm was measured every 30 minutes for an hour (beginning at 5 minutes).

Results

Approximately 80% of the liquid urokinase activity was recovered for samples irradiated to 45 kGy in the presence of at least about 120 mM ascorbate. Increasing the ascorbate concentration above about 300 mM resulted in increased absorbance at 405-620 nm for both the 0 and 45 kGy samples. For the 0 kGy samples, there was a slight decrease in absorbance at 405-620 nm with increasing concentrations of ascorbate up to about 200 mM.

### Example 4

In this experiment, the effect of gamma irradiation on LUK in glass vials, microwell modules and 0.2 ml PCR tubes was evaluated.

Methods

Samples of Sigma LUK were reconstituted to 10,000 IU/ml in Sigma reconstitution buffer (16 mM Tris, pH 7.5, 90 mM NaCl) containing 200 mM sodium ascorbate. Aliquots of 150 μl were placed either in glass vials, microwell modules or 0.2 ml PCR tubes. Duplicate samples were either stored or irradiated at 4° C. to a total dose of about 40 kGy. The standard UK assay was performed.

Results

The LUK activity after irradiation to 40 kGy in glass vials, microwell modules and 0.2 ml PCR tubes was 70%, 68% and 65%, respectively, when comparing assays at 1500 μM substrate. Little change in either Vₘₐₓ or Kₘ during storage in the different containers occurred. After irradiation to 40 kGy, Vₘₐₓ dropped by about 50% for LUK in vials and tubes, but 64% of control remained for the samples in microwells. Similarly, after irradiation to 40 kGy, Kₘ was about 80% of control for microwells, while Kₘ for vials and tubes dropped by about 50%.

### Example 5

In this experiment, the effects of gamma radiation on Sigma LUK in the presence or absence of various stabilizers were evaluated.

Methods

Samples of liquid Sigma LUK at 5000 IU/ml, in total volume of a 200 μl/well, were irradiated to a total dose of about 40 kGy in the presence or absence of 250 mM sodium ascorbate alone, or 200 mM ascorbate+Trolox and/or Urate at pH 7.5. Samples were prepared in polystyrene microwell modules with polyethylene stoppers. Samples were analyzed by assaying in a volume of 200 μl with 500 IU/ml LUK and 1500 μM substrate. The assay temperature was 32° C. All samples were assayed in duplicate on the same plate.

Results

The presence of 250 mM of ascorbate increased the LUK activity after irradiation to 40 kGy from about 12% to about 65% of control. Increasing the level of sodium ascorbate up to 1 M increased the remaining activity up to about 74%. After 40 kGy irradiation of LUK, 27.5K IU/ml, lyophilized in the presence of 200 mM ascorbate, 300 μM Urate and 400 μM Trolox, 88% of the control activity remained.

### Example 6

In this experiment, the protective effect of the dipeptide stabilizer L-carnosine, alone or in combination with sodium ascorbate (50 mM), on gamma irradiated liquid urokinase was evaluated.

Methods

Liquid urokinase samples (2000 IU/ml) were prepared using a buffer solution containing 100 mM Tris pH 8.8, 100 mM NaCl, and 0.2% PEG 8000. Samples were irradiated at a dose rate of 1.92 kGy/hr to a total dose of 45 kGy at 4° C.

Urokinase activity was determined using a calorimetric assay. The substrate was Urokinase Substrate I, Colorimetric, CalBiochem 672157 lot B23901. Substrate was reconstituted in a buffer solution containing 50 mM Tris pH 8.8, 50 mM NaCl and 0.1% PEG 8000 to a concentration of 1 mM). Irradiated samples were centrifuged (1-1.5×1000 RPM, Sorvall RT6000B Refrigerated Centrifuge with Sorvall rotor H1000B) for approximately 3 minutes and then 50 μl of substrate solution were added. The samples with added substrate were incubated at 37° C. with shaking and absorbance at 406-620 nm determined at 20 minute intervals beginning 5 minutes after addition of substrate to the sample.

Results

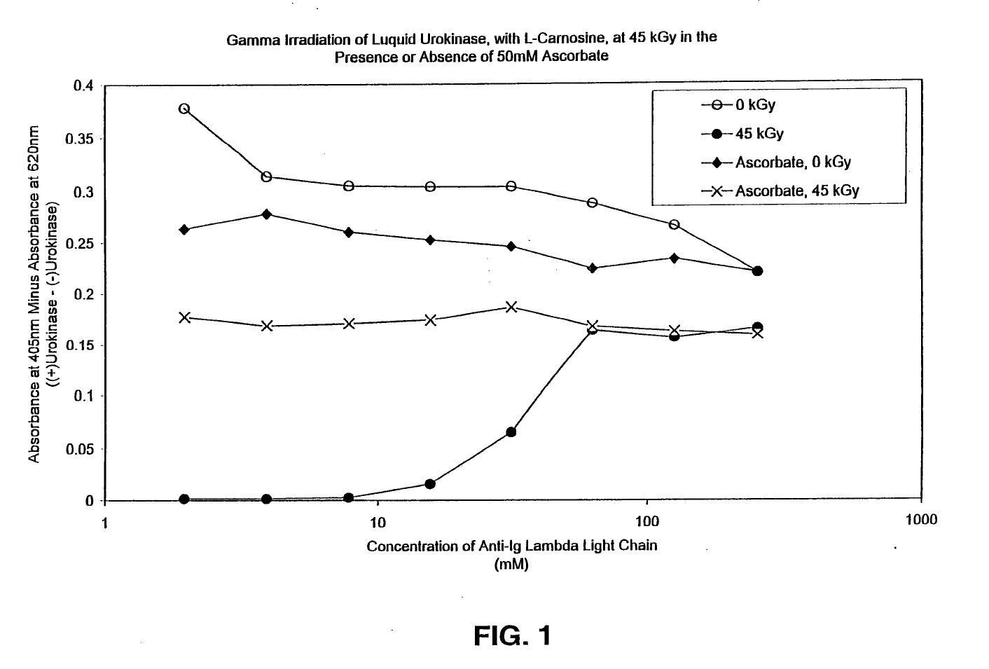

As shown in FIG. 1, L-carnosine showed a concentration dependent protection of liquid urokinase (from about 15 mM to about 62.5 mM) irradiated to a total dose of 45 kGy. At concentrations greater than 62.5 mM, no additional protective effect was observed. When L-carnosine was combined with ascorbate (50 mM), a protective effect on irradiated liquid urokinase was also observed.

### Example 7

In this experiment, the protective effect of the dipeptide stabilizer anserine on gamma irradiated liquid urokinase was evaluated.

Methods

Liquid urokinase samples (2000 IU/ml) were prepared using a buffer solution containing 100 mM Tris pH 8.8, 100 mM NaCl, and 0.2% PEG 8000. Samples were irradiated at a dose rate of 1.92 kGy/hr to a total dose of 45 kGy at 4° C.

Urokinase activity was determined using a colorimetric assay. The substrate was Urokinase Substrate I, Colorimetric, CalBiochem 672157 lot B23901. Substrate was reconstituted in a buffer solution containing 50 mM Tris pH 8.8, 50 mM NaCl and 0.1% PEG 8000 to a concentration of 1 mM). Irradiated samples were centrifuged (1-1.5×1000 RPM, Sorvall RT6000B Refrigerated Centrifuge with Sorvall rotor H1000B) for approximately 3 minutes and then 50 μl of substrate solution were added. The samples with added substrate were incubated at 37° C. with shaking and absorbance at 406-620 nm determined at 20 minute intervals beginning 5 minutes after addition of substrate to the sample.

Results

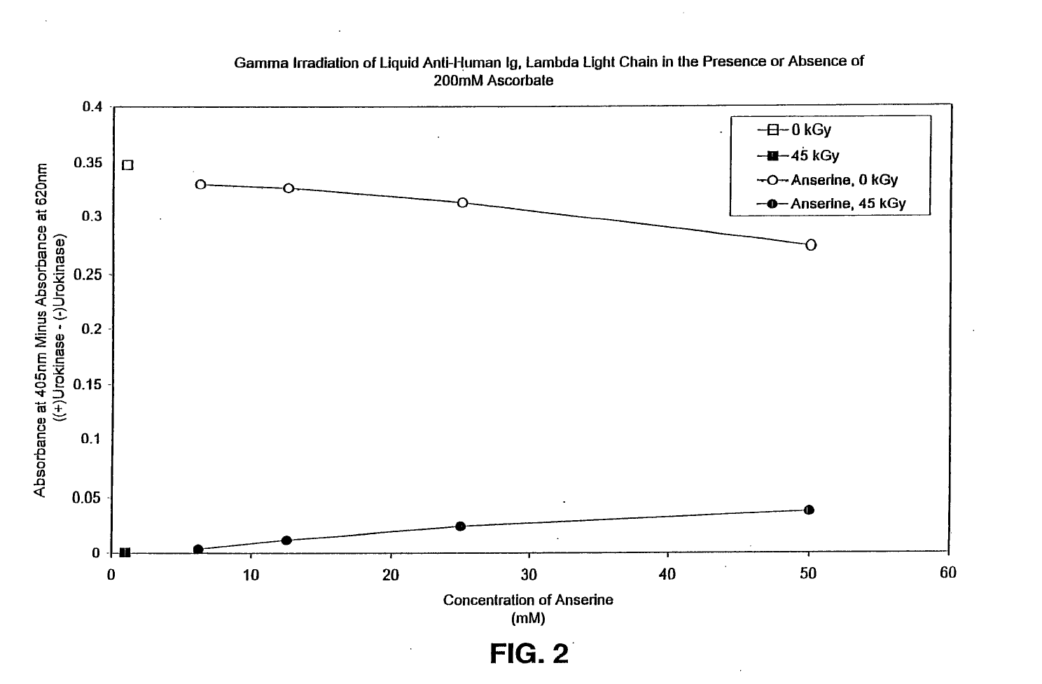

As shown in FIG. 2, the addition of anserine provided approximately 10-15% protection to liquid urokinase irradiated to a total dose of 45 kGy. In contrast, liquid urokinase samples containing no anserine showed a complete loss of activity.

### Example 8

In this experiment, the protective effect of L-carnosine on gamma irradiated liquid urokinase was evaluated.

Methods

Liquid urokinase samples (2000 IU/ml) were prepared using a buffer solution containing 100 mM Tris pH 8.8, 100 mM NaCl, and 0.2% PEG 8000. Samples were irradiated at a dose rate of 1.92 kGy/hr to a total dose of 45 kGy at 4° C.

Urokinase activity was determined using a colorimetric assay. The substrate was Urokinase Substrate I, Colorimetric, CalBiochem 672157 lot B23901. Substrate was reconstituted in a buffer solution containing 50 mM Tris pH 8.8, 50 mM NaCl and 0.1% PEG 8000 to a concentration of 1 mM). Irradiated samples were centrifuged (1-1.5×1000 RPM, Sorvall RT6000B Refrigerated Centrifuge with Sorvall rotor H1000B) for approximately 3 minutes and then 50 μl of substrate solution were added. The samples with added substrate were incubated at 37° C. with shaking and absorbance at 406-620 nm determined at 20 minute intervals beginning 5 minutes after addition of substrate to the sample.

Results

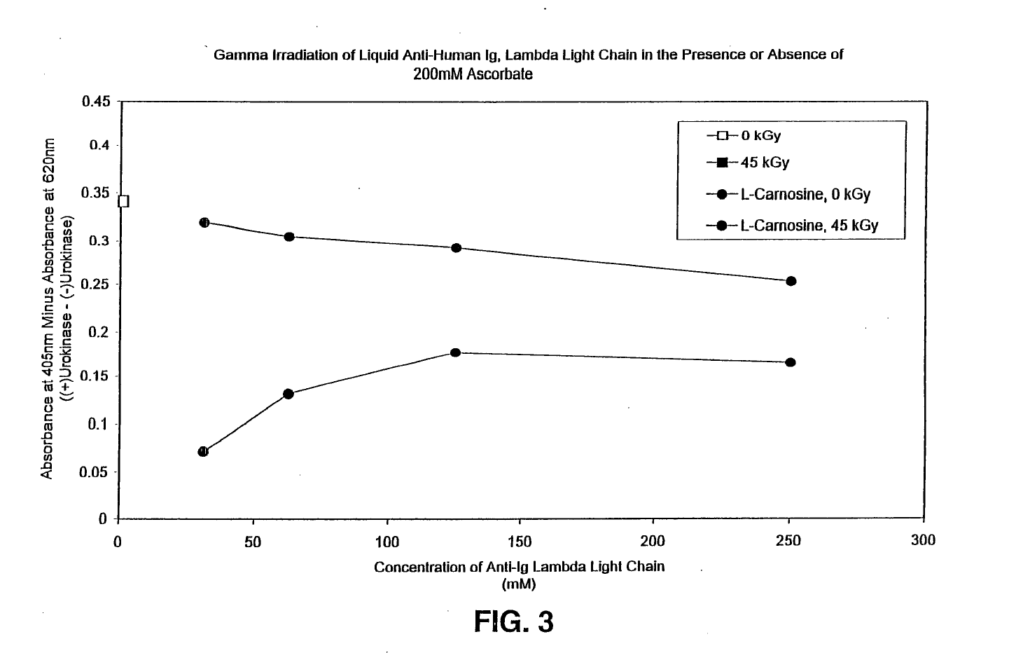

As shown in FIG. 3, L-carnosine showed a concentration dependent protection of liquid urokinase irradiated to a total dose of 45 kGy. At concentrations of 125 and 250 mM, L-carnosine protected approximately 60-65% of the activity of irradiated liquid urokinase.

### Example 9

In this experiment, the effect of gamma radiation on dried urokinase suspended in polypropylene glycol (PPG) 400 or phosphate buffered saline (PBS) was evaluated.

Methods

Six 1.5 ml polypropylene microfuge tubes containing urokinase and PPG400 (tubes 2 and 5), PBS (tubes 3 and 6) or dry urokinase alone (tubes 1 and 4) were prepared as indicated in the table below. Tubes 4-6 were gamma irradiated at 45 kGy (1.9 kGy/hr) at 4° C. Tubes 1-3 were controls (4° C.).

| | | | volume | volume |
|---|---|---|---|---|
| | | weight of dry | PPG400 | PBS |
| Tube | Sample | urokinase (mg) | (μl) | (μl) |
| 1 | dry urokinase alone | 3.2 | 0 | 0 |
| 2 | urokinase suspended in | 3.16 | 126 | 0 |
| | PPG400 | | | |
| 3 | urokinase suspended in PBS | 3.08 | 0 | 123 |
| 4 | dry urokinase alone | 3.38 | 0 | 0 |
| 5 | urokinase suspended in | 3.3 | 132 | 0 |
| | PPG400 | | | |
| 6 | urokinase suspended in PBS | 3.52 | 0 | 141 |

After irradiation, the samples were centrifuged at room temperature for 5 minutes at 14k RPM. PPG400 solvent was removed from tubes 2 and 5 and 120 μl PBS were added to those two tubes. 128 μl and 135 μl PBS were added to tubes 1 and 4, respectively (urokinase concentration of 40,000 IU/ml). All samples were then diluted 50-fold with PBS and absorbance at 280 nm was determined. 50 μl of each diluted sample were then added to a 96-well microtiter plate, followed by 50 μl of 3 mM substrate in 2× assay buffer. The plates were incubated at 37° C. with shaking and absorption read at both 405 and 620 nm every 20 minutes beginning 5 minutes after substrate addition. The absorption at 630 nm (background) was subtracted from the value at 405 nm to obtain a corrected absorption value. The final concentration of urokinase was 1000 IU/ml.

Results

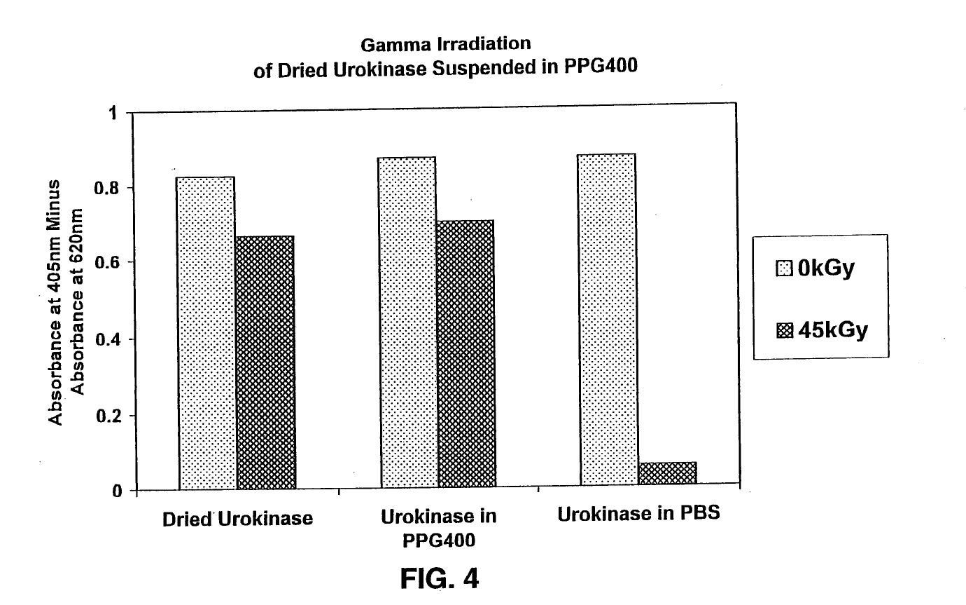

As shown in FIG. 4, Urokinase suspended in PPG400 and then gamma irradiated to a total dose of 45 kGy maintained the same percent activity as gamma irradiated dry powder urokinase (80%). In contrast, urokinase suspended in PBS subjected to the same gamma irradiation maintained only 6% activity.

### Example 10

In this experiment, the protective effects of the combination of ascorbate and trolox and the combination of ascorbate, trolox and urate on urokinase enzymatic activity were evaluated as a function of pH in phosphate buffer solution.

Methods

Samples were prepared in 2 ml vials, each containing 1,000 IU of urokinase (Sigma) and 35 μl of 1M phosphate buffer (pH=4, 5, 5.5, 6.0, 6.47, 7, 7.5, 7.8, 8.5 or 9.0). Stabilizers (a mixture of 100 μl of 3 mM trolox and 100 μl of 2 M sodium ascorbate or a mixture of 100 μl of 3 mM trolox, 100 μl of 2 M sodium ascorbate and 100 μl of 3 mM sodium urate) or trolox alone were added and the samples gamma irradiated to 45 kGy at a dose rate of 1.8 kGy/hr at 4° C. Residual urokinase activity was determined at room temperature at 5 and 25 minutes after commencement of reaction by addition of urokinase colorimetric substrate #1 (CalBiochem). Optical densities were measured at 405 nm, with subtraction of the optical density at 620 nm.

Results

The irradiated samples containing a stabilizer exhibited much greater retention of urokinase activity compared to samples containing only a single stabilizer across the range of pH tested. More specifically, at pH 4, irradiated samples containing trolox/ascorbate (T/A) retained 65.1% of urokinase activity and samples containing trolox/ascorbate/urate (T/A/U) retained 66.2% of urokinase activity. In contrast, at pH 4, samples containing only trolox retained only 5.3% of urokinase activity. The following results were also obtained:

| pH | stabilizer | urokinase activity |
|---|---|---|
| 5.0 | trolox | 13% |
| | T/A | 72.2% |
| | T/A/U | 62.2% |
| 5.5 | trolox | 13% |
| | T/A | 66.7% |
| | T/A/U | 66.3% |
| 6.0 | trolox | 30% |
| | T/A | 61.8% |
| | T/A/U | 61.8% |
| 6.47 | trolox | 30% |
| | T/A | 70.5% |
| | T/A/U | 70.2% |
| 7.0 | trolox | 20% |
| | T/A | 69.5% |
| | T/A/U | 65.9% |
| 7.5 | trolox | 24% |
| | T/A | 72.1% |
| | T/A/U | 64.0% |
| 7.8 | trolox | 28% |
| | T/A | 63.5% |
| | T/A/U | 70.7% |
| 8.5 | trolox | 23% |
| | T/A | 64.4% |
| | T/A/U | 70.2% |
| 9.0 | trolox | 38% |
| | T/A | 71.3% |
| | T/A/U | 68.73% |

### Example 11

In this experiment, the protective effects of the combination of ascorbate and urate on urokinase enzymatic activity were evaluated as a function of pH in phosphate buffer solution.

Methods

Samples were prepared in 2 ml vials, each containing 1,000 IU of urokinase (Sigma) and 35 μl of 1M phosphate buffer (pH=4, 5, 6.0, 6.47, 7, 7.8 or 9.0). A stabilizer of 100 μl of 2 M sodium ascorbate and 100 μl of 3 mM sodium urate was added and the samples gamma irradiated to 45 kGy at a dose rate of 1.8 kGy/hr at 4° C. Residual urokinase activity was determined at room temperature at 5 and 25 minutes after commencement of reaction by addition of urokinase colorimetric substrate #1 (CalBiochem). Optical densities were measured at 405 nm, with subtraction of the optical density at 620 nm.

Results

The irradiated samples containing a stabilizer exhibited much greater retention of urokinase activity compared to samples containing only urate across the range of pH tested. More specifically, irradiated samples containing ascorbate/urate retained between 48.97% (at pH 9.0) and 64.01% (at pH 6.47) of urokinase activity, whereas irradiated samples containing only urate retained essentially no urokinase activity.

### Example 12

In this experiment, the effects of gamma radiation on urokinase (LUK) either frozen or lyophilized in the presence or absence of ascorbate containing PPV or vaccinia were evaluated.

Method

Samples of LUK in the presence or absence of 200 mM ascorbate were prepared. The samples were either frozen or lyophilized and irradiated to a total dose of about 50 kGy with gamma radiation. Control samples were not irradiated (0 kGy). Samples were then assayed for structural integrity and viral inactivation.

Results

Viral inactivation for frozen and freeze dried samples is shown in the following table:

| | | | Vₘₐₓ | | | Viral Kill, | |
|---|---|---|---|---|---|---|---|
| | | Kₘ, μM | Abs | % Recovery | | Logs | |
| | Sample | [substrate] | (405-620 nm) | (Vₘₐₓ, 50/0) | R² | Vaccinia | PPV |
| Frozen | LUK/0 | 280.5 +/− 35.5 | 0.2757 +/− 0.0124 | — | 0.99217908 | — | — |
| Solid | LUK/50 | 187.0 +/− 29.5 | 0.1000 +/− 0.0050 | 36.3% | 0.98569685 | >4.55 | 5.83 |
| | LUK + Asc/0 | 400.9 +/− 25.7 | 0.4098 +/− 0.0104 | — | 0.99825533 | — | — |
| | LUK + Asc/50 | 352.1 +/− 27.2 | 0.3366 +/− 0.0099 | 82.1% | 0.99729097 | 3.61 | 4.95 |
| Freeze | LUK/0 | 238.9 +/− 46.1 | 0.2126 +/ 0.0140 | — | 0.98081096 | — | — |
| Dried | LUK/50 | 189.8 +/− 44.7 | 0.1012 +/ 0.0076 | 47.6% | 0.97002458 | >4.95 | 3.44 |
| | LUK + Asc/0 | 349.8 +/− 28.9 | 0.3645 +/ 0.0115 | — | 0.99694532 | — | |
| | LUK + Asc/50 | 341.9 +/− 22.1 | 0.3343 +/ 0.0081 | 91.7% | 0.99810530 | >4.14 | 2.68 |

For both frozen and lyophilized samples, samples containing ascorbate showed higher urokinase activity than samples not containing ascorbate. The absolute signal (Vₘₐₓ, Kₘ) produced by urokinase following gamma irradiation was nearly identical for lyophilized and frozen samples in the presence or absence of ascorbate.

Having now fully described this invention, it will be understood to those of ordinary skill in the art that the methods of the present invention can be carried out with a wide and equivalent range of conditions, formulations, and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

1. A method for sterilizing a preparation of urokinase that is sensitive to radiation, said method comprising irradiating said preparation of urokinase with radiation for a time effective to sterilize said preparation of urokinase at a rate effective to sterilize said preparation of urokinase and to protect said preparation of urokinase from said radiation.

2. A method for sterilizing a preparation of urokinase that is sensitive to radiation, said method comprising:
(i) applying to said preparation of urokinase at least one stabilizing process selected from the group consisting of:
(a) adding to said preparation of urokinase at least one stabilizer;
(b) reducing the residual solvent content of said preparation of urokinase;
(c) reducing the temperature of said preparation of urokinase;
(d) reducing the oxygen content of said preparation of urokinase;
(e) adjusting or maintaining the pH of said preparation of urokinase; and
(f) adding to said preparation of urokinase at least one non-aqueous solvent; and
(ii) irradiating said preparation of urokinase with a suitable radiation at an effective rate for a time effective to sterilize said preparation of urokinase, wherein said at least one stabilizing process protects said preparation of urokinase from said radiation.

3. A method for sterilizing a preparation of urokinase that is sensitive to radiation, said method comprising irradiating said preparation of urokinase with radiation to a total dose effective to sterilize said preparation of urokinase at a rate effective to sterilize said preparation of urokinase and to protect said preparation of urokinase from said radiation.

4. The method according to claim 2, wherein said residual solvent is an organic solvent.

5. The method according to claim 2, wherein said residual solvent is an aqueous solvent.

6. The method according to claim 2, wherein said stabilizing process and said rate are together effective to protect said preparation of urokinase from said radiation.

7. The method according to claim 2, wherein at least two stabilizing processes are applied and said at least two stabilizing processes are together effective to protect said preparation of urokinase from said radiation.

8. The method according to claim 1, 2 or 3, wherein said effective rate comprises a rate of not more than 3.0 kGy/hour.

9. The method according to claim 1, 2 or 3, wherein said effective rate comprises a rate of not more than 2.5 kGy/hr.

10. The method according to claim 1, 2 or 3, wherein said effective rate comprises a rate of not more than 2.0 kGy/hr.

11. The method according to claim 1, 2 or 3, wherein said effective rate comprises a rate of not more than 1.0 kGy/hr.

12. The method according to claim 1, 2 or 3, wherein said effective rate comprises a rate of not more than 0.3 kGy/hr.

13. The method according to claim 1, 2 or 3, wherein said effective rate comprises a rate of more than 3.0 kGy/hour.

14. The method according to claim 1, 2 or 3, wherein said effective rate comprises a rate of at least 5.0 kGy/hour.

15. The method according to claim 1, 2 or 3, wherein said effective rate comprises a rate of at least 18.0 kGy/hour.

16. The method according to claim 1, 2 or 3, wherein said effective rate comprises a rate of at least 30.0 kGy/hour.

17. The method according to claim 1, 2 or 3, wherein said effective rate comprises a rate of at least 45 kGy/hour.

18. The method according to claim 1, 2 or 3, wherein said preparation of urokinase is maintained in a low oxygen atmosphere.

19. The method according to claim 1, 2 or 3, wherein said preparation of urokinase is maintained in an atmosphere comprising at least one noble gas or nitrogen.

20. The method according to claim 19, wherein said noble gas is argon.

21. The method according to claim 1, 2 or 3, wherein said preparation of urokinase is maintained in a vacuum.

22. The method according to claim 2, wherein said residual solvent content is reduced by a method selected from the group consisting of lyophilization, drying, concentration, addition of solute, evaporation, chemical extraction, spray-drying and vitrification.

23. The method according to claim 2, wherein said residual solvent content is less than about 15%.

24. The method according to claim 2, wherein said residual solvent content is less than about 10%.

25. The method according to claim 2, wherein said residual solvent content is less than about 3%.

26. The method according to claim 2, wherein said residual solvent content is less than about 2%.

27. The method according to claim 2, wherein said residual solvent content is less than about 1%.

28. The method according to claim 2, wherein said residual solvent content is less than about 0.5%.

29. The method according to claim 2, wherein said residual solvent content is less than about 0.08%.

30. The method according to claim 1, 2 or 3, wherein at least one sensitizer is added to said preparation of urokinase prior to said step of irradiating said preparation of urokinase.

31. The method according to claim 2, wherein said at least one stabilizer is an antioxidant.

32. The method according to claim 2, wherein said at least one stabilizer is a free radical scavenger.

33. The method according to claim 2, wherein said at least one stabilizer is a ligand.

34. The method according to claim 33, wherein said ligand is heparin.

35. The method according to claim 2, wherein said at least one stabilizer reduces damage due to reactive oxygen species.

36. The method according to claim 2, wherein said at least one stabilizer is selected from the group consisting of: ascorbic acid or a salt or ester thereof; glutathione; vitamin E or a derivative thereof; albumin; sucrose; glycylglycine; L-carnosine; cysteine; silymarin; diosmin; hydroquinonesulfonic acid; 6-hydroxy-2,5,7,8-tetramethylchroman-2-carboxylic acid; uric acid or a salt or ester thereof; methionine; histidine; N-acetyl cysteine; lipoic acid; sodium formaldehyde sulfoxylate; gallic acid or a derivative thereof; propyl gallate; ethanol; acetone; rutin; epicatechin; biacalein; purpurogallin; pyruvate; lactate; and mixtures of two or more thereof.

37. The method according to claim 36, wherein said mixtures of two or more stabilizers are selected from the group consisting of: mixtures of ethanol and acetone; mixtures of ascorbic acid, or a salt or ester thereof, and uric acid, or a salt or ester thereof; mixtures of ascorbic acid, or a salt or ester thereof, and 6-hydroxy-2,5,7,8-tetramethylchroman-2-carboxylic acid; mixtures of ascorbic acid, or a salt or ester thereof, uric acid, or a salt or ester thereof, and 6-hydroxy-2,5,7,8-tetramethylchroman-2-carboxylic acid; mixtures of ascorbic acid, or a salt or ester thereof, uric acid, or a salt or ester thereof, 6-hydroxy-2,5,7,8-tetramethylchroman-2-carboxylic acid, and albumin; mixtures of ascorbic acid, or a salt or ester thereof, uric acid, or a salt or ester thereof, 6-hydroxy-2,5,7,8-tetramethylchroman-2-carboxylic acid, albumin and sucrose; mixtures of ascorbic acid, or a salt or ester thereof, and glycylglycine; mixtures of ascorbic acid, or a salt or ester thereof, glycylglycine and albumin; mixtures of ascorbic acid, or a salt or ester thereof, and L-carnosine; mixtures of ascorbic acid, or a salt or ester thereof, and cysteine; mixtures of ascorbic acid, or a salt or ester thereof, and N-acetyl cysteine; mixtures of ascorbic acid, or a salt or ester thereof, uric acid, or a salt or ester thereof, 6-hydroxy-2,5,7,8-tetramethylchroman-2-carboxylic acid, and silymarin; mixtures of ascorbic acid, or a salt or ester thereof, uric acid, or a salt or ester thereof, 6-hydroxy-2,5,7,8-tetramethylchroman-2-carboxylic acid, and diosmin; mixtures of ascorbic acid, or a salt or ester thereof, uric acid, or a salt or ester thereof, and lipoic acid; mixtures of ascorbic acid, or a salt or ester thereof, uric acid, or a salt or ester thereof, and hydroquinonesulfonic acid; mixtures of pyruvate and lactate; mixtures of pyruvate and ascorbate, or a salt or ester thereof; mixtures of pyruvate and histidine; and mixtures of uric acid, or a salt or ester thereof, lipoic acid, sodium formaldehyde sulfoxylate, gallic acid, or a derivative thereof, propyl gallate, and 6-hydroxy-2,5,7,8-tetramethylchroman-2-carboxylic acid.

38. The method according to claim 2, wherein said at least one stabilizer is a dipeptide stabilizer.

39. The method according to claim 38, wherein said dipeptide stabilizer is selected from the group consisting of glycyl-glycine (Gly-Gly), carnosine and anserine.

40. The method according to claim 2, wherein said at least one stabilizer inhibits the generation of free radicals.

41. The method according to claim 2, wherein said at least one stabilizer inhibits the generation of reactive oxygen species.

42. The method according to claim 2, wherein said at least one stabilizer is present in a concentration of at least 200 mM.

43. The method according to claim 2, wherein said at least one stabilizer is present in a concentration of at least 1 mM.

44. The method according to claim 2, wherein said at least one stabilizer is present in a concentration of at least 2 mM.

45. The method according to claim 2, wherein said at least one stabilizer is present in a concentration of at least 5 mM.

46. The method according to claim 2, wherein said at least one stabilizer is present in a concentration of at least 10 mM.

47. The method according to claim 2, wherein said at least one stabilizer is present in a concentration of at least 25 mM.

48. The method according to claim 2, wherein said at least one stabilizer is present in a concentration of at least 50 mM.

49. The method according to claim 2, wherein said at least one stabilizer is present in a concentration of at least 100 mM.

50. The method according to claim 2, wherein said at least one stabilizer is present in a concentration of from 0.1 mM to 10 mM.

51. The method according to claim 2, wherein said at least one stabilizer is present in a concentration of from 0.1 mM to 50 mM.

52. The method according to claim 2, wherein said at least one stabilizer is present in a concentration of from 10 mM to 100 mM.

53. The method according to claim 2, wherein said at least one stabilizer is present in a concentration of from 10 mM to 50 mM.

54. The method according to claim 2, wherein said at least one stabilizer is present in a concentration of from 50 mM to 100 mM.

55. The method according to claim 1, 2 or 3, wherein the temperature of said preparation of urokinase is about ambient temperature at the initiation of said irradiation.

56. The method according to claim 1, 2 or 3, wherein the temperature of said preparation of urokinase is below ambient temperature at the initiation of said irradiation.

57. The method according to claim 1, 2 or 3, wherein the temperature of said preparation of urokinase is below the freezing point of said preparation of urokinase at the initiation of said irradiation.

58. The method according to claim 1, 2 or 3, wherein the temperature of said preparation of urokinase is below the eutectic point of said preparation of urokinase at the initiation of said irradiation.

59. The method according to claim 1, 2 or 3, wherein the temperature of said preparation of urokinase is above ambient temperature at the initiation of said irradiation.

60. The method according to claim 1, 2 or 3, wherein the temperature of said preparation of urokinase is at least −75° C. at the initiation of said irradiation.

61. The method according to claim 1, 2 or 3, wherein the temperature of said preparation of urokinase is at least −70° C. at the initiation of said irradiation.

62. The method according to claim 1, 2 or 3, wherein the temperature of said preparation of urokinase is at least −55° C. at the initiation of said irradiation.

63. The method according to claim 1, 2 or 3, wherein the temperature of said preparation of urokinase is at least −50° C. at the initiation of said irradiation.

64. The method according to claim 1, 2 or 3, wherein the temperature of said preparation of urokinase is at least −40° C. at the initiation of said irradiation.

65. The method according to claim 1, 2 or 3, wherein the temperature of said preparation of urokinase is at least −30° C. at the initiation of said irradiation.

66. The method according to claim 1, 2 or 3, wherein the temperature of said preparation of urokinase is at least −20° C. at the initiation of said irradiation.

67. The method according to claim 1, 2 or 3, wherein the temperature of said preparation of urokinase is at least −10° C. at the initiation of said irradiation.

68. The method according to claim 1, 2 or 3, wherein the temperature of said preparation of urokinase is at least 4° C. at the initiation of said irradiation.

69. The method according to claim 1, 2 or 3, wherein the temperature of said preparation of urokinase is about the same temperature as dry ice at the initiation of said irradiation.

70. The method according to claim 1, 2 or 3, wherein the temperature of said preparation of urokinase is between −10° C. and −55° C. at the initiation of said irradiation.

71. The method according to claim 1, 2 or 3, wherein the temperature of said preparation of urokinase is between −10° C. and −50° C. at the initiation of said irradiation.

72. The method according to claim 1, 2 or 3, wherein the temperature of said preparation of urokinase is between −10° C. and −30° C. at the initiation of said irradiation.

73. The method according to claim 1, 2 or 3, wherein the temperature of said preparation of urokinase is between −30° C. and −70° C. at the initiation of said irradiation.

74. The method according to claim 1, 2 or 3, wherein the temperature of said preparation of urokinase is between −30° C. and −50° C. at the initiation of said irradiation.

75. The method according to claim 1, 2 or 3, wherein the temperature of said preparation of urokinase is between −40° C. and −55° C. at the initiation of said irradiation.

76. The method according to claim 1, 2 or 3, wherein the temperature of said preparation of urokinase is between −40° C. and −50° C. at the initiation of said irradiation.

77. The method according to claim 1, 2 or 3, wherein the functional activity of said preparation of urokinase after sterilization by irradiation is about 100% of the pre-irradiation value.

78. The method according to claim 1, 2 or 3, wherein the functional activity of said preparation of urokinase after sterilization by irradiation is greater than 100% of the pre-irradiation value.

79. The method according to claim 1, 2 or 3, wherein the functional activity of said preparation of urokinase after sterilization by irradiation is at least 95% of the pre-irradiation value.

80. The method according to claim 1, 2 or 3, wherein the functional activity of said preparation of urokinase after sterilization by irradiation is about 90% of the pre-irradiation value.

81. The method according to claim 1, 2 or 3, wherein the functional activity of said preparation of urokinase after sterilization by irradiation is about 85% of the pre-irradiation value.

82. The method according to claim 1, 2 or 3, wherein the functional activity of said preparation of urokinase after sterilization by irradiation is about 80% of the pre-irradiation value.

83. The method according to claim 1, 2 or 3, wherein the functional activity of said preparation of urokinase after sterilization by irradiation is about 70% of the pre-irradiation value.

84. The method according to claim 1, 2 or 3, wherein the functional activity of said preparation of urokinase after sterilization by irradiation is about 60% of the pre-irradiation value.

85. The method according to claim 1, 2 or 3, wherein the functional activity of said preparation of urokinase after sterilization by irradiation is about 50% of the pre-irradiation value.

86. The method according to claim 1, 2 or 3, wherein said preparation of urokinase is irradiated to a total dose of at least 105 kGy.

87. The method according to claim 1, 2 or 3, wherein said preparation of urokinase is irradiated to a total dose of at least 90 kGy.

88. The method according to claim 1, 2 or 3, wherein said preparation of urokinase is irradiated to a total dose of at least 80 kGy.

89. The method according to claim 1, 2 or 3, wherein said preparation of urokinase is irradiated to a total dose of at least 70 kGy.

90. The method according to claim 1, 2 or 3, wherein said preparation of urokinase is irradiated to a total dose of at least 65 kGy.

91. The method according to claim 1, 2 or 3, wherein said preparation of urokinase is irradiated to a total dose of at least 55 kGy.

92. The method according to claim 1, 2 or 3, wherein said preparation of urokinase is irradiated to a total dose of at least 50 kGy.

93. The method according to claim 1, 2 or 3, wherein said preparation of urokinase is irradiated to a total dose of at least 40 kGy.

94. The method according to claim 1, 2 or 3, wherein said preparation of urokinase is irradiated to a total dose of at least 30 kGy.

95. The method according to claim 1, 2 or 3, wherein said preparation of urokinase is irradiated to a total dose of at least 25 kGy.

96. The method according to claim 1, 2 or 3, wherein said preparation of urokinase is irradiated to a total dose of at least 10 kGy.

97. The method according to claim 1, 2 or 3, wherein said preparation of urokinase has a pH of less than 7.

98. The method according to claim 1, 2 or 3, wherein said preparation of urokinase has a pH of less than 6.

99. The method according to claim 1, 2 or 3, wherein said preparation of urokinase has a pH of less than 5.

100. The method according to claim 1, 2 or 3, wherein said preparation of urokinase has a pH of less than 4.

101. The method according to claim 1, 2 or 3, wherein said preparation of urokinase has a pH of less than 3.

102. The method according to claim 1, 2 or 3, wherein said preparation of urokinase has a pH of less than 2.

103. The method according to claim 1, 2 or 3, wherein said preparation of urokinase has a pH of less than 1.

104. The method according to claim 1, 2 or 3, wherein said preparation of urokinase is a mammalian preparation of urokinase.

105. The method according to claim 1, 2 or 3, wherein said preparation of urokinase is a bovine preparation of urokinase.

106. The method according to claim 1, 2 or 3, wherein said preparation of urokinase is an ovine preparation of urokinase.

107. The method according to claim 1, 2 or 3, wherein said preparation of urokinase is a porcine preparation of urokinase.

108. The method according to claim 1, 2 or 3, wherein said preparation of urokinase is an equine preparation of urokinase.

109. The method according to claim 1, 2 or 3, wherein said preparation of urokinase is a caprine preparation of urokinase.

110. The method according to claim 1, 2 or 3, wherein said preparation of urokinase is obtained from a fetal, immature or adult mammal.

112. The method according to claim 1, 2 or 3, wherein said preparation of urokinase contains at least one biological contaminant or pathogen selected from the group consisting of viruses, bacteria, yeasts, molds, fungi, parasites and prions or similar agents responsible, alone or in combination, for TSEs.

113. The method according to claim 1, 2 or 3, wherein said effective rate is constant throughout said irradiation.

114. The method according to claim 1, 2 or 3, wherein said effective rate is not constant throughout said irradiation.

115. The method according to claim 114, wherein said effective rate is less than 3.0 kGy/hr for at least a portion of said irradiation.

116. The method according to claim 1, 2 or 3, wherein said irradiating is performed under conditions whereby the temperature of said preparation of urokinase increases during said irradiating from an initial temperature (Tᵢ) to a final temperature (T_{f}) and further wherein said increase in the temperature of said preparation of urokinase (ΔT) is about equal to the total dose of said radiation (D) divided by the specific heat constant of said preparation of urokinase (c).

117. The method according to claim 116, wherein said final temperature (T_{f}) is at or below a temperature effective to protect said preparation of urokinase from said radiation.

118. The method according to claim 116, wherein said increase in the temperature of said preparation of urokinase (ΔT) is about 0.25° C./kGy.

119. The method according to claim 2, wherein said residual solvent content is reduced by the addition of an effective amount of at least one solute.

120. The method according to claim 2, wherein said residual solvent content is reduced by lyophilization.

121. The method according to claim 1, 2 or 3, wherein the temperature of said preparation of urokinase is about ambient temperature for at least a portion of said irradiation.

122. The method according to claim 1, 2 or 3, wherein the temperature of said preparation of urokinase is below ambient temperature for at least a portion of said irradiation.

123. The method according to claim 1, 2 or 3, wherein the temperature of said preparation of urokinase is below the freezing point of said preparation of urokinase for at least a portion of said irradiation.

124. The method according to claim 1, 2 or 3, wherein the temperature of said preparation of urokinase is below the eutectic point of said preparation of urokinase for at least a portion of said irradiation.

125. The method according to claim 1, 2 or 3, wherein the temperature of said preparation of urokinase is above ambient temperature for at least a portion of said irradiation.

126. The method according to claim 1, 2 or 3, wherein the temperature of said preparation of urokinase is at least −75° C. for at least a portion of said irradiation.

127. The method according to claim 1, 2 or 3, wherein the temperature of said preparation of urokinase is at least −70° C. for at least a portion of said irradiation.

128. The method according to claim 1, 2 or 3, wherein the temperature of said preparation of urokinase is at least −55° C. for at least a portion of said irradiation.

129. The method according to claim 1, 2 or 3, wherein the temperature of said preparation of urokinase is at least −50° C. for at least a portion of said irradiation.

130. The method according to claim 1, 2 or 3, wherein the temperature of said preparation of urokinase is at least −40° C. for at least a portion of said irradiation.

131. The method according to claim 1, 2 or 3, wherein the temperature of said preparation of urokinase is at least −30° C. for at least a portion of said irradiation.

132. The method according to claim 1, 2 or 3, wherein the temperature of said preparation of urokinase is at least −20° C. for at least a portion of said irradiation.

133. The method according to claim 1, 2 or 3, wherein the temperature of said preparation of urokinase is at least −10° C. for at least a portion of said irradiation.

134. The method according to claim 1, 2 or 3, wherein the temperature of said preparation of urokinase is at least 4° C. for at least a portion of said irradiation.

135. The method according to claim 1, 2 or 3, wherein the temperature of said preparation of urokinase is about the same temperature as dry ice for at least a portion of said irradiation.

136. The method according to claim 1, 2 or 3, wherein the temperature of said preparation of urokinase is between −10° C. and −55° C. for at least a portion of said irradiation.

137. The method according to claim 1, 2 or 3, wherein the temperature of said preparation of urokinase is between −10° C. and −50° C. for at least a portion of said irradiation.

138. The method according to claim 1, 2 or 3, wherein the temperature of said preparation of urokinase is between −10° C. and −30° C. for at least a portion of said irradiation.

139. The method according to claim 1, 2 or 3, wherein the temperature of said preparation of urokinase is between −30° C. and −70° C. for at least a portion of said irradiation.

140. The method according to claim 1, 2 or 3, wherein the temperature of said preparation of urokinase is between −30° C. and −50° C. for at least a portion of said irradiation.

141. The method according to claim 1, 2 or 3, wherein the temperature of said preparation of urokinase is between −40° C. and −55° C. for at least a portion of said irradiation.

142. The method according to claim 1, 2 or 3, wherein the temperature of said preparation of urokinase is between −40° C. and −50° C. for at least a portion of said irradiation.

143. The method according to claim 2, wherein said residual solvent content of said preparation of urokinase is reduced to a level between 0.5% and 5.0% prior to said irradiation.

144. The method according to claim 2, wherein said residual solvent content of said preparation of urokinase is reduced to a level between 0.5% and 2.5% prior to said irradiation.

145. The method according to claim 2, wherein said residual solvent content of said preparation of urokinase is reduced to a level between 1.0% and 10.0% prior to said irradiation.

146. The method according to claim 2, wherein said residual solvent content of said preparation of urokinase is reduced to a level between 2.5% and 10.0% prior to said irradiation.

147. The method according to claim 2, wherein said at least one stabilizer comprises at least one α-keto acid.

148. The method according to claim 1, 2 or 3, wherein the temperature of said preparation of urokinase is below the glass transition temperature of said preparation of urokinase at the initiation of said irradiation.

149. The method according to claim 1, 2 or 3, wherein said preparation of urokinase is obtained from a mammal.

150. The method according to claim 8, wherein said effective rate further comprises a rate of more than 3.0 kGy/hour.

151. The method according to claim 9, wherein said effective rate further comprises a rate of more than 2.5 kGy/hr.

152. The method according to claim 10, wherein said effective rate further comprises a rate of more than 2.0 kGy/hr.

153. The method according to claim 11, wherein said effective rate further comprises a rate of more than 1.0 kGy/hr.

154. The method according to claim 12, wherein said effective rate further comprises a rate of more than 0.3 kGy/hr.

155. The method according to claim 8, wherein said effective rate further comprises a rate of at least 5.0 kGy/hour.

156. The method according to claim 8, wherein said effective rate further comprises a rate of at least 18.0 kGy/hour.

157. The method according to claim 8, wherein said effective rate further comprises a rate of at least 30.0 kGy/hour.

158. The method according to claim 8, wherein said effective rate further comprises a rate of at least 45 kGy/hour.

159. The method according to claim 9, wherein said effective rate further comprises a rate of at least 5.0 kGy/hour.

160. The method according to claim 9, wherein said effective rate further comprises a rate of at least 18.0 kGy/hour.

161. The method according to claim 9, wherein said effective rate further comprises a rate of at least 30.0 kGy/hour.

162. The method according to claim 9, wherein said effective rate further comprises a rate of at least 45 kGy/hour.

163. The method according to claim 10, wherein said effective rate further comprises a rate of at least 5.0 kGy/hour.

164. The method according to claim 10, wherein said effective rate further comprises a rate of at least 18.0 kGy/hour.

165. The method according to claim 10, wherein said effective rate further comprises a rate of at least 30.0 kGy/hour.

166. The method according to claim 10, wherein said effective rate further comprises a rate of at least 45 kGy/hour.

167. The method according to claim 11, wherein said effective rate further comprises a rate of at least 5.0 kGy/hour.

168. The method according to claim 11, wherein said effective rate further comprises a rate of at least 18.0 kGy/hour.

169. The method according to claim 11, wherein said effective rate further comprises a rate of at least 30.0 kGy/hour.

170. The method according to claim 11, wherein said effective rate further comprises a rate of at least 45 kGy/hour.

171. The method according to claim 12, wherein said effective rate further comprises a rate of at least 5.0 kGy/hour.

172. The method according to claim 12, wherein said effective rate further comprises a rate of at least 18.0 kGy/hour.

173. The method according to claim 12, wherein said effective rate further comprises a rate of at least 30.0 kGy/hour.

174. The method according to claim 12, wherein said effective rate further comprises a rate of at least 45 kGy/hour.

175. The method according to claim 1, 2 or 3, wherein said effective rate is not constant.

176. The method according to claim 1 or 2, wherein said effective rate is not constant for at least a portion of said time effective to sterilize said preparation of urokinase.
